(19) United States
(12) Patent Application Publication
(10) Pub. No.: US 2009/0234621 A1
(43) Pub. Date: Sep. 17, 2009

(54) METHOD OF OPTIMIZING PARAMETERS IN THE ENTIRE PROCESS OF ANALYSING A DNA CONTAINING SAMPLE AND METHOD OF MODELING SAID PROCESS

(75) Inventors: Peter Gill, Birmingham (GB); James Curran, Birmingham (GB)

Correspondence Address: MERCHANT & GOULD PC, P.O. BOX 2903, MINNEAPOLIS, MN, 55402-0903, US

(73) Assignee: FORENSIC SCIENCE SERVICE LIMITED, Birmingham (GB)

(21) Appl. No.: 11/720,721

(22) PCT Filed: Dec. 5, 2005
(86) PCT No.: PCT/GB05/04641

§ 371 Date: May 28, 2008

(30) Foreign Application Priority Data
Dec. 3, 2004 (GB) 0426579.9
Apr. 1, 2005 (GB) 0506673.3

(51) Int. Cl. G06F 17/10; G06G 7/48; G06F 19/00; G01N 33/48

(52) U.S. Cl. 703/2; 703/11

(57) ABSTRACT

A method of optimizing one or more parameters in a process for considering a DNA containing sample using a method of modeling and a method of modeling itself are provided. The method of modeling the process for considering a DNA containing sample uses a graphical model. The model seeks to provide one or more optimized parameters for the consideration process. The methods aim to consider the whole process, for instance, the number of cells required for the process and/or the extraction efficiency and/or the sub-sample volume relative to the sample volume and/or the amplification efficiency and/or the optimum number of amplification cycles and/or the effect of degradation on the amount of amplifiable DNA in the sample.

The present invention concerns improvements in and relating to the DNA consideration process, particularly, but not exclusively in relation to the simulation of the DNA consideration process.

Some attempts have been made to simulate or model that part of the DNA consideration process involving PCR. These attempts have used specific probability approaches and have considered a part of the process in isolation.

The invention has amongst its potential aims to simulate the DNA consideration process. The invention has amongst its potential aims to provide a quick and cost effective source of DNA consideration process data.

According to a first aspect the present invention provides a method of modeling a process for considering a DNA containing sample, the process being modeled by a graphical model.

The method of modeling may include simulating the process. The method may model or simulate one or more parts of the process. Preferably the method models or simulates all parts of the process.

The process for considering the DNA containing sample may comprise one or more parts. Extraction from the sample to provide an extracted sample may be a part of the process. Selection of a sub-sample of the sample, particularly from an extracted sample may be a part of the process. The sub-sample may be an aliquot. Amplification of a sub-sample, particularly by PCR, to give an amplified product may be a part of the process. Electrophoresis of a sub-sample, particularly the amplified product or a part thereof may be a part of the process. Analysis of a sub-sample, particularly after electrophoreis, may be a part of the process. The analysis may include allocation of allele designations as a part of the process.

The DNA containing sample may be from a single source and/or multiple sources. The sample may be from a male and/or female source. The sample may be from one or more unknown sources and/or be from one or more known sources. The sample may be a mixture of DNA from more than one source. The sample may contain haploid and/or diploid cells. The sample may contain sperm and/or epithelial cells. The sample may contain degraded DNA.

The graphical model may be a Bayes net. The graphical model may be formed of one or more nodes and one or more directed edges. Preferably the directed edges extend between nodes. Preferably a directed edge between two nodes reflects the dependence of one on the other.

The graphical model may represent one or more of the parts of the process by a node. One or more constant nodes may be provided. Preferably all constant nodes are starter nodes. Preferably no constant nodes have parent nodes. One or more stochastic nodes may be provided. Preferably stochastic nodes are given a distribution. Stochastic nodes may be parent and/or child nodes. Preferably each part of the process is represented by a node. A node may represent a parameter, such as an input and/or output parameter. The node may further represent a distribution, preferably a probability distribution. The graphical model preferably represent the dependencies between parts of the process, preferably between nodes, ideally through the use of links.

The model may take into account one or more parameters. The parameters may be input parameters and/or output parameters. One or more of the parameters may be the number of cells in the sample. One or more of the parameters may be the proportion of the sample extracted into an extracted sample by the process. One or more of the parameters may particularly be the extraction efficiency. One or more of the parameters may be the volume of the sub-sample relative to the volume of the sample the sub-sample is taken from. One or more of the parameters may be the amplification efficiency. One or more of the parameters may particularly be the fraction of the amplifiable molecules amplified in each cycle of PCR. One or more of the parameters may be the number of cycles of amplification, particularly the number of PCR cycles. The number may be 28 or 34 cycles. The aforementioned parameters may particularly be considered input parameters. The parameters now mentioned may be considered output parameters. One or more of the parameters may be the probability of allele dropout. One or more of the parameters may be the number of molecules of one or more of the alleles of interest after amplification. One or more of the parameters may be the ratio of the number of molecules of one allele compared with another for a locus. One or more of the parameters may be the heterozygous balance.

The method may be used to model one or more further parts of the process. The method may be used to model allele dropout. The method may used to model allele dropout due to the absence of one or more allele types from the sample and/or extracted sample and/or sub-sample. The method may, alternatively or additionally, be used to model allele dropout due to one or more allele types being below the detectable level in the amplification product. The method may be used to model allele dropout due to stochastic effects, particularly in small DNA samples. The method may be used to model allele dropout due to degradation of the sample, particularly the DNA therein.

The method may take into account the size of the DNA fragment being amplified and/or investigated and/or analyised when modeling for degradation, particularly where two or more different size fragments are being considered. The chance of degradation may vary with size. The chance of degradation may assume a function with size. The function may have a transition point or point of inflexion, for instance where the rate of change in the chance of degradation with size changes rapidly. The transition point and/or point of inflexion may be between 100 and 160 bases, preferably between 110 and 140 bases, more preferably between 120 and 130 bases and ideally 125 bases +/−1 base. A higher chance of degradation may be applied to fragments whose size is above a threshold than to those below it. The threshold may be set at a value between 100 and 160 bases, preferably between 110 and 140 bases, more preferably between 120 and 130 bases and ideally 125 bases +/−1 base. The chance of degradation may be provided with at a first level for a first fragment length, with a second level being applied to a second fragment length, preferably a second fragment length which adjoins the first fragment length. A third level may be provided for a third fragment length. Preferably the third fragment length adjoins the second fragment length. The third fragment length and the first fragment length may be the same length. The chance of degradation for the first and third fragment lengths may be the same. The chance of degradation may be lower for the first and/or third lengths than for the second length. A fourth fragment length may be provided intermediate the first and second fragment lengths. A fifth fragment length may be provided intermediate the second and third fragment lengths. The fourth and fifth fragments may be of the same length and/or have the same chance of degradation. The fourth and/or fifth fragments may have a chance of degradation which is intermediate that of the first and/or third fragments compared with the second fragment. The fourth and/or fifth fragments may have a chance of degradation which is higher than the first and/or third fragments and/or which is lower than the second fragment.

The method may be used to model stutter. The method may model stutter as only being possible during amplification.

The method may be used to model contamination.

Preferably the method uses binomial theory to model one or more parts of the process. The binomial theory may be of the form Bin (n, π), where n is the number of template molecules for the part of the process and π is an efficiency parameter between 0-1 for that part of the process.

The method may be provided in or be performed by an expert system. The method may be performed by a computer. The method may be provided as a MATLAB program. The program may be rewritten into C++. Any computer program can be used

Preferably the method models the entire process for considering the DNA containing sample.

The method may be used to assess one or more parameters in the process. The method may be used to measure one or more parameters in the process. The method may be used to determine, preferably optimize, one or more parameters in the process.

The method may be used to determine the number of cells required for the process, particularly the number of cells required to ensure that all the alleles in the sample are represented in the extracted sample and/or aliquot and/or amplification product, ideally in respect of a heterozygote locus. The number of cells may be expressed relative to a confidence level. The method may be used to determine the effect of variation in the number of cells on the process or one or more parts thereof.

The method may be used to determine the extraction efficiency. The method may be used to determine the effect of variation in the extraction efficiency on the process or one or more parts thereof.

The method may be used to determine the sub-sample volume relative to the sample volume. The method may be used to vary the volume of the sub-sample volume compared with the sample volume from a first proposed value, such as that normally used in the process, to a revised value, preferably a value sufficiently high to avoid dropout. The method may be used to determine the effect of variation in the sub-sample volume to sample volume on the process or one or more parts thereof.

The method may be used to determine the amplification efficiency. The method may be used to determine the effect of variations in amplification efficiency on the process.

The method may be used to determine the optimum number of amplification cycles, particularly the number necessary to provide a number of molecules in excess of a threshold number in the amplified sample. The method may be used to determine the effect of variation in the number of amplification cycles on the process or one or more parts thereof.

The method may be used to determine the effect of degradation on the amount of amplifiable DNA in the sample. The amount of amplifiable DNA determined may be used to decide on one or more parameters for a subsequent analysis, such as the analysis method and/or amplification cycle number and/or aliquot.

The method may include determining the effect of one or more of the parameters on one or more of the other parameters.

The method may include obtaining and/or obtaining an estimate of one or more of the parameters by physical analysis. The method may include comparing the value of a parameter obtained by physical analysis with the value of that parameter obtained by modeling.

The method may further include the part of quantification. This part may follow the extraction and precede the selection of the sub-sample and/or amplification. The method may include modeling quantification. The modeling of the quantification may be used to give the suggested sub-sample volume to sample volume and/or the suggested number of amplification cycles.

The method may be used to model across a plurality of loci.

The method may be used to model one or more test scenarios. The one or more test scenarios may consider the different results possible with a given set of parameters. Thus the method may be used to model the effect of probability on the one or more test scenarios. One or more test scenarios may be modeled before the process is applied to a physical sample. The process may be modified in one or more ways as a result of the modeling. One or more of the parameters may be modified. The modification may take place compared with one or more normal processes or protocols therefore. The method may be used to mock up the effect of the process on a sample.

The method may be used to model one or more different processes, for instance a process under development. A process may be modified as a result of the modeling. The process may be modified in terms of one or more parts of that process. The process may be modified by changing a part and/or adding a part and/or removing a part.

The method may be used to model a process, with the results of the modeling being provided to an expert system. The results may be used to investigate the expert system. The results may be used to modify the expert system. The results may be used to develop the expert system. The method may be integrated into existing expert systems by estimating parameters on a case by case basis

The method may be used to model a process, with the results being used to consider the extremes of the results arising. The results may be used to modify the process to make it more applicable to those extremes.

The first aspect of the invention may include any of the features, options or possibilities set out elsewhere in this application.

According to a second aspect the present invention provides a method of modeling a process for considering a DNA containing sample, the process being of one or more parts, one or more of the parts being modeled using binomial theory.

The second aspect of the invention may include any of the features, options or possibilities set out elsewhere in this application.

According to a third aspect the present invention provides a method of modeling a process for considering a DNA containing sample, the process being of a number of parts, the method including providing the model with the number of cells that the sample contains, an efficiency for the extraction from that sample into an extraction sample, a proportion that a sub-sample volume represents compared with the extraction sample volume, a number of amplification cycles and an efficiency for the amplification of the sub-sample.

The third aspect of the invention may include any of the features, options or possibilities set out elsewhere in this application.

According to a fourth aspect the present invention provides a method of modeling a process for considering a DNA containing sample, the process being formed of one or more parts, the method determining the value or range of values of a parameter of one of those parts.

Preferably the method is applied to a plurality of different processes. Preferably the plurality of different processes are assessed against one another and/or compared with one another, preferably using the parameter.

The fourth aspect of the invention may include any of the features, options or possibilities set out elsewhere in this application.

According to a fifth aspect the present invention provides a method of modeling a process for considering a DNA containing sample, the method of modeling producing data of the same type as is produced by the process.

The data may be used as a substitute for and/or in addition to data obtained from the physical analysis of samples. The data may be used to test and/or develop and/or modifying other systems. The systems may be expert systems. The data may be used to test the effect of changes in one or more of the parameters of the system.

The model may be modified to accept data from and/or provide data to one or more other systems. The model may be modified to handle parameters from and/or provide parameters for one or more other systems.

The fifth aspect of the invention may include any of the features, options or possibilities set out elsewhere in this application.

According to a sixth aspect of the invention we provide a method of designing an analysis technique for determining the identity of one or more targets within a DNA sample, one or more of the DNA targets being investigated using a fragment of DNA associated with the target, wherein the targets are selected so as to be determinable using fragments of less than a threshold size and/or wherein the fragments are selected so as to be less than a threshold size.

The sixth aspect of the invention may include any of the features, options or possibilities set out elsewhere in this application, particularly from those in and/or following the seventh aspect of the invention.

According to a seventh aspect of the invention we provide a method of analyzing a sample to determine the identity of one or more targets within a DNA sample, one or more of the DNA targets being investigated using a fragment of DNA associated with the target, wherein the targets are selected so as to be determinable using fragments of less than a threshold size and/or wherein the fragments are selected so as to be less than a threshold size.

Preferably the threshold size is a size below which DNA is preferentially protected against degradation, particularly compared with larger sizes. The preferential protection against degradation may be due to the DNA being wrapped around one or more histone proteins, preferably an octomer of histone proteins. The threshold size may be the size of a complete turn of the DNA about a histone core, +/−22 bases. The threshold size may be between 100 and 160 bases, preferably between 110 and 140 bases, more preferably between 120 and 130 bases and ideally 125 bases +/−1 base.

The method of analysis may be concerned with STR's and/or STR's and/or SNP's.

The seventh aspect of the invention may include any of the features, options or possibilities set out elsewhere in this application

According to an eighth aspect of the invention we provide a method of quantifying the amount of DNA in a sample and/or the amount of DNA in a sample from a particular source, using an amplicon and/or a fragment and/or a fragment associated with a target and/or an amplified sequence of a threshold size or greater.

The threshold size may be a size below which DNA is preferentially protected against degradation, particularly compared with larger sizes. The preferential protection against degradation may be due to the DNA being wrapped around one or more histone proteins, preferably an octomer of histone proteins. The threshold size may be the size of a complete turn of the DNA about a histone core, +/−22 bases. The threshold size may be between 100 and 160 bases, preferably between 110 and 140 bases, more preferably between 120 and 130 bases and ideally 125 bases +/−1 base. The threshold size may be a size equal to or greater than 100 bases, more preferably equal to or greater than 110 bases still more preferably equal to or greater than 120 bases and ideally 125 bases or more.

The method may include using one or more further amplicons and/or a fragments and/or a fragments associated with targets and/or an amplified sequences. One or more of these may be of a first size. The first size may be between 50 and 70 bases, preferably between 60 and 66 bases and ideally may be 62 bases or 64 bases. One or more of these may be of a second size. The second size may be between 160 bases and 300 bases, preferably between 175 bases and 250 bases, more preferably between 190 and 210 bases. The second size may be at least 160 bases, preferably at least 175 bases and more preferably at least 190 bases.

The quantification method may consider the amount of an identifier unit, such as a dye, particularly a fluorescent dye, observable with each cycle of amplification. The identifier unit may be a part of a probe, preferably together with a quencher. The probe is preferably cleaved during extension, ideally to separate the identifier unit and quencher have a first

The method of analysis may be concerned with STR's and/or STR's and/or SNP's.

The method may consider male DNA and/or female DNA. Differences in the extent of degradation may be established between the male and female DNA.

The eighth aspect of the invention may include any of the features, options or possibilities set out elsewhere in this application

According to a ninth aspect of the invention we provide a method of investigating the extent of degradation of DNA in a sample, the method including using an amplicon and/or a fragment and/or a fragment associated with a target and/or an amplified sequence of a first size and using an amplicon and/or a fragment and/or a fragment associated with a target and/or an amplified sequence of a threshold size or greater.

Preferably the method includes considering the variation in the quantity of DNA suggested by the first size compared with the amount suggested by the size of the threshold size or greater. The closer the two quantities are to one another the less degradation assumed to have occurred. The method may include using one or more further sizes to quantify the amount of DNA and so inform on the extent of degradation.

The threshold size may be a size below which DNA is preferentially protected against degradation, particularly compared with larger sizes. The preferential protection against degradation may be due to the DNA being wrapped around one or more histone proteins, preferably an octomer of histone proteins. The threshold size may be the size of a complete turn of the DNA about a histone core, +/−22 bases. The threshold size may be between 100 and 160 bases, preferably between 110 and 140 bases, more preferably between 120 and 130 bases and ideally 125 bases +/−1 base. The threshold size may be a size equal to or greater than 100 bases, more preferably equal to or greater than 110 bases still more preferably equal to or greater than 120 bases and ideally 125 bases or more.

The first size may be between 50 and 70 bases, preferably between 60 and 66 bases and ideally may be 62 bases or 64 bases.

The method may include using one or more further amplicons and/or a fragments and/or a fragments associated with targets and/or an amplified sequences. One or more of these may be of a second size. The second size may be between 160 bases and 300 bases, preferably between 175 bases and 250 bases, more preferably between 190 and 210 bases. The second size may be at least 160 bases, preferably at least 175 bases and more preferably at least 190 bases.

The ninth aspect of the invention may include any of the features, options or possibilities set out elsewhere in this application

Various embodiments of the present invention will now be described, by way of example only, and with reference to the accompanying drawings in which

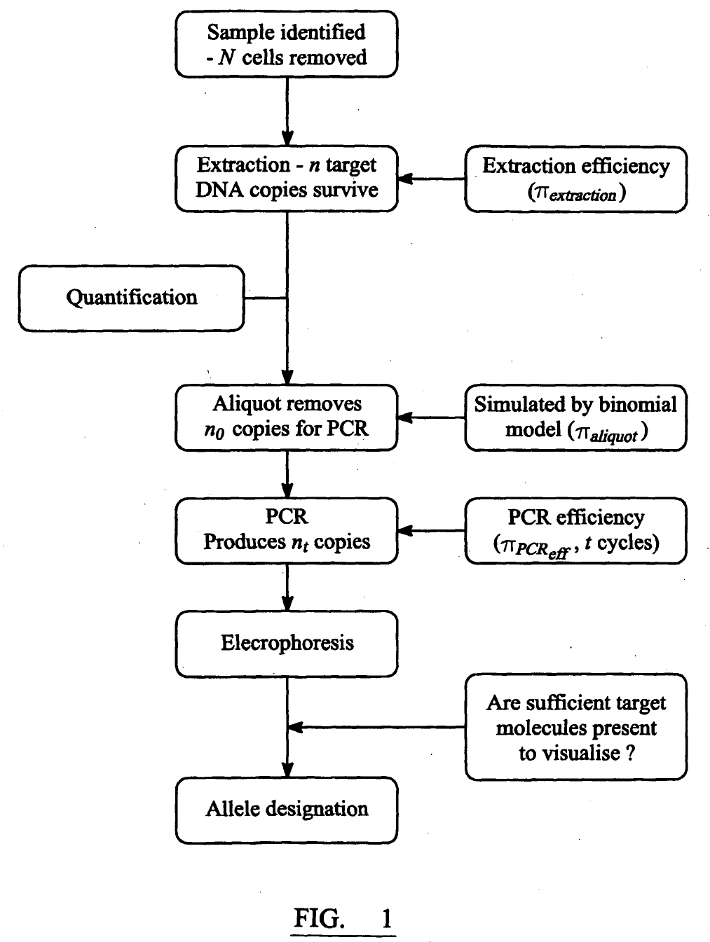

FIG. 1 is an overview of the DNA consideration process;

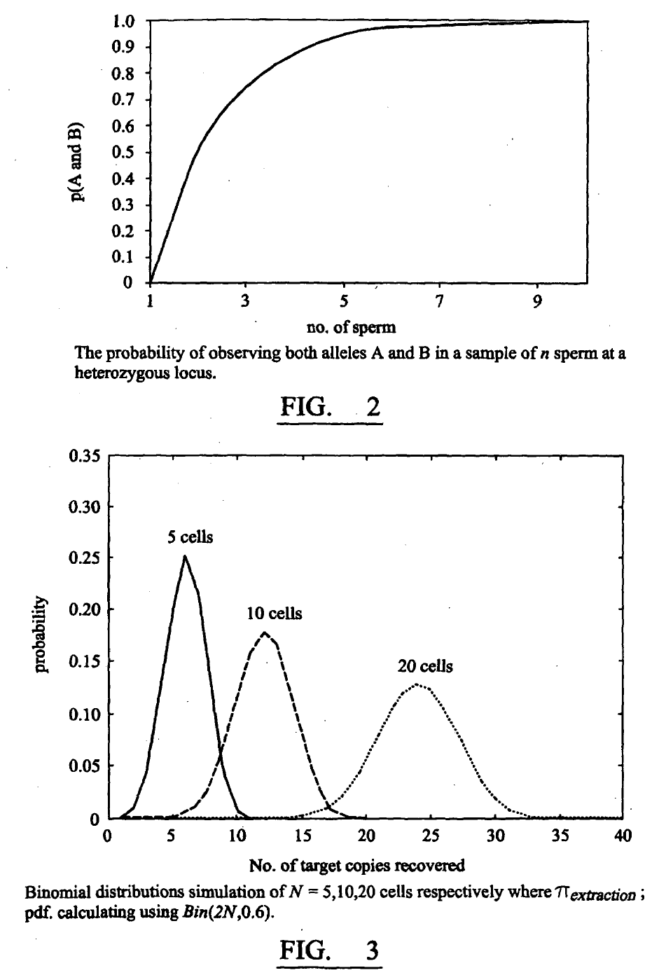

FIG. 2 illustrates the probability of observing both alleles A and B in a sample of n sperm at a heterozygous locus;

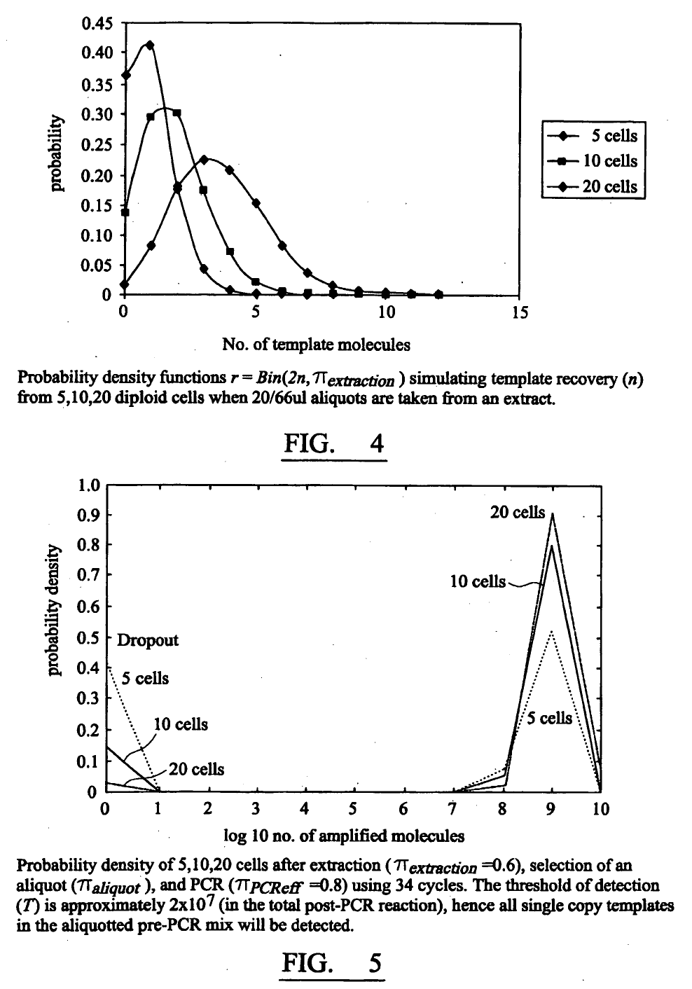

FIG. 3 illustrates binomial distributions simulation of N=5, 10, 20 cells respectively, using Bin(2N, π_{extraction}); π_{extraction}=a number from 0-1—typical extraction efficiency may be 60% or 0.6.

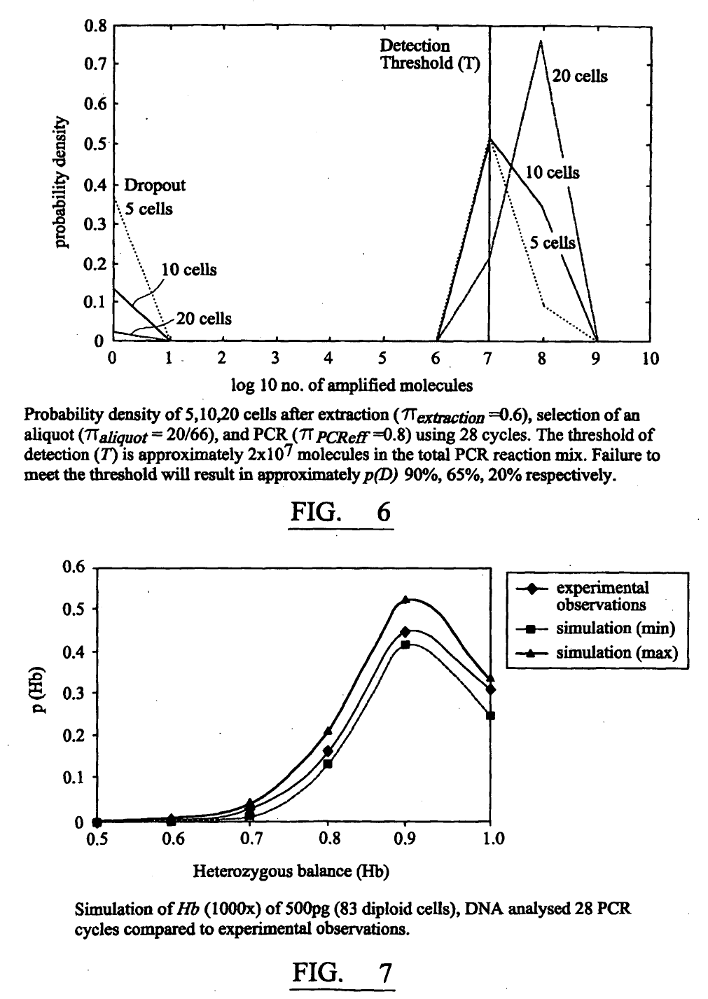

FIG. 4 illustrates probability density functions r=Bin(2N, π_{aliquot}) simulating template recovery (n) from 5, 10, 20 diploid cells when 20/66 ul aliquots are taken from an extract, other aliquot proportions could be considered similarly;

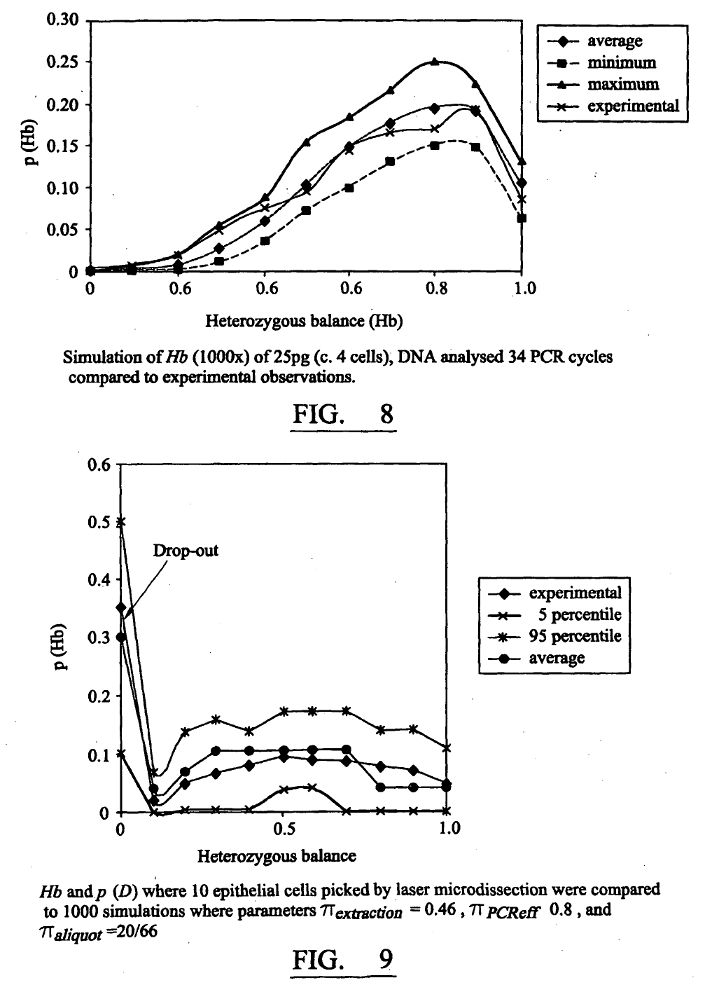

FIG. 5 is a plot of probability density of 5, 10, 20 cells after extraction (π_{extraction}=0.6), selection of an aliquot (π_{aliquot}=20/66), and PCR (PCR_{ef}=0.8) using 34 cycles—the threshold of detection (T) is approximately 2×10⁷ (in the total post-PCR reaction), hence all single copy templates in the aliquotted pre-PCR mix will be detected; note that T is dependent upon the equipment used to detect the PCR fluorescent products

FIG. 6 is a plot of probability density of 5, 10, 20 cells after extraction (π_{extraction}=0.6), selection of an aliquot (π_{Aliquot}=20/66), and PCR (π_{PCReff}=0.8) using 28 cycles—the threshold of detection (T) is approximately 2×10⁷ molecules in the total PCR reaction mix—failure to meet the threshold will result in approximately p(D) 90%, 65%, 20% respectively;

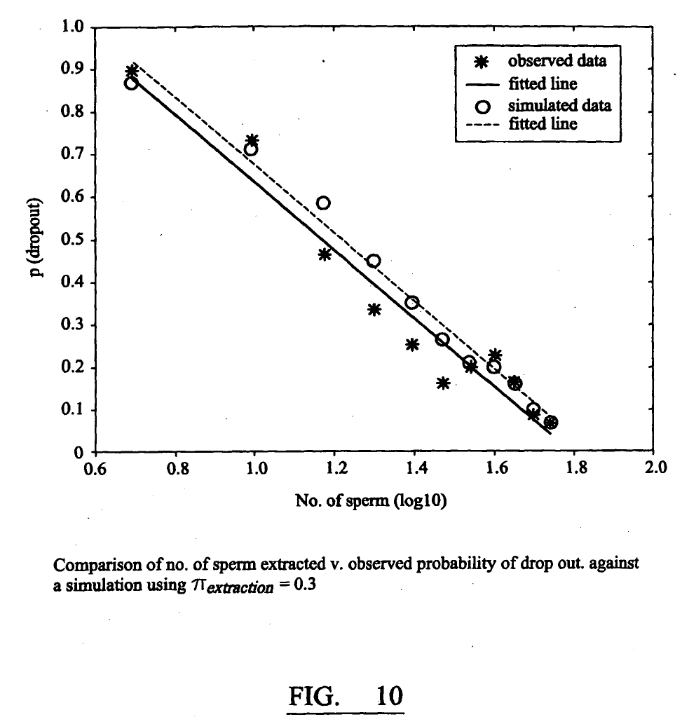

FIG. 7 is a simulation of Hb (10000×) of 500 pg (83 diploid cells), DNA analysed 28 PCR cycles compared to experimental observations;

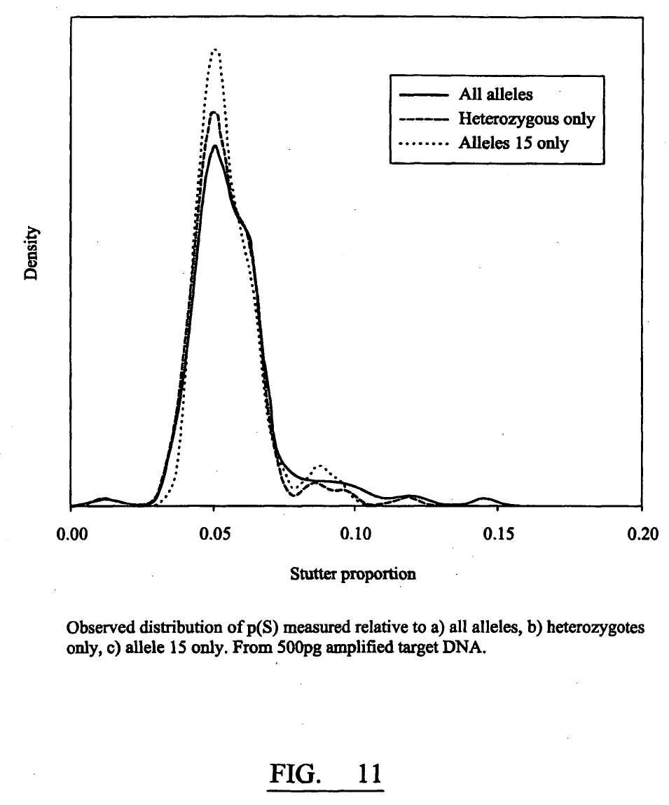

FIG. 8 is a simulation of Hb (1000×) of 25 pg DNA (c. 4 cells), DNA analysed 34 PCR cycles compared to experimental observations;

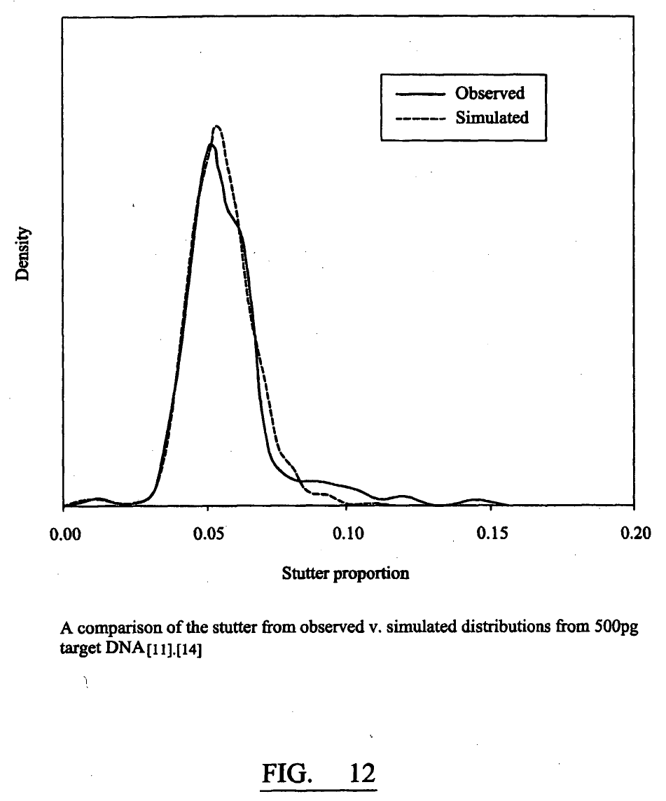

FIG. 9 illustrates Hb and p(D), where 10 epithelial cells picked by laser microdissection were compared to 1000 simulations where parameters π_{extraction}=0.46, π_{PCReff}=0.8, π_{Aliquot}=20/66;

FIG. 10 shows a comparison of no. of sperm extracted v. observed probability of drop out. against a simulation using π_{extraction}=0.3;

FIG. 11 shows observed distribution of p(S) measured relative to a) all alleles, b) heterozygotes only c) allele 15 only—from 500 pg amplified target DNA;

FIG. 12 is a comparison of the stutter from observed v. simulated distributions from 500 pg target DNA;

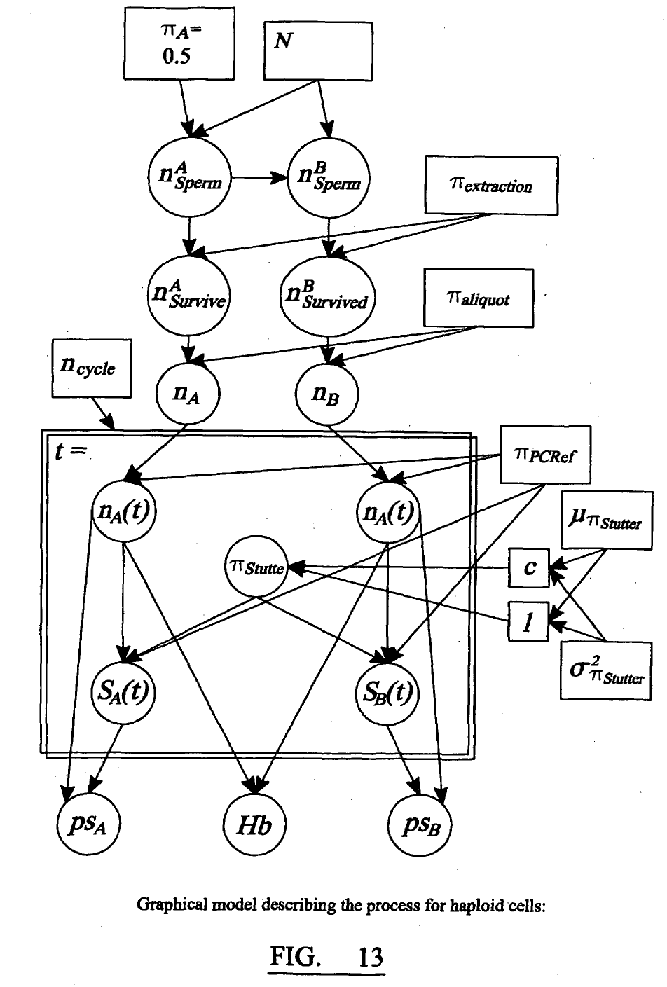

FIG. 13 is a graphical model describing the process according to an embodiment of the invention, for haploid cells;

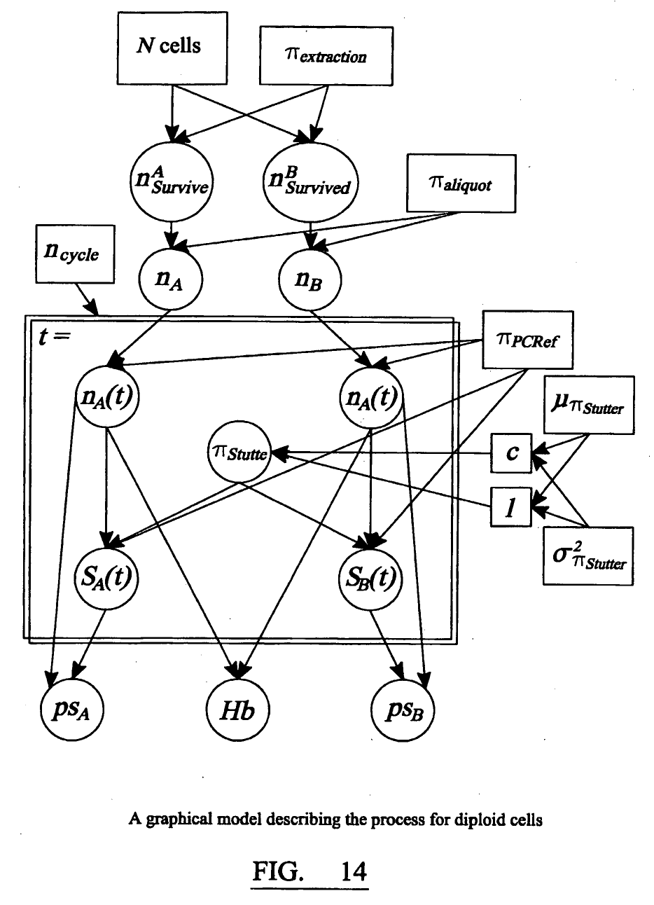

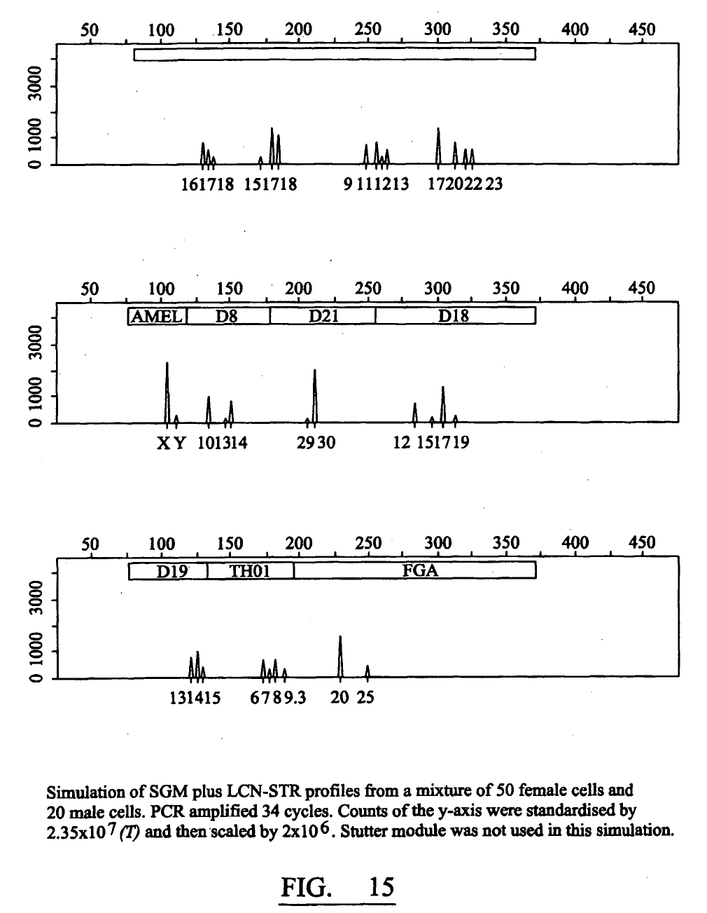

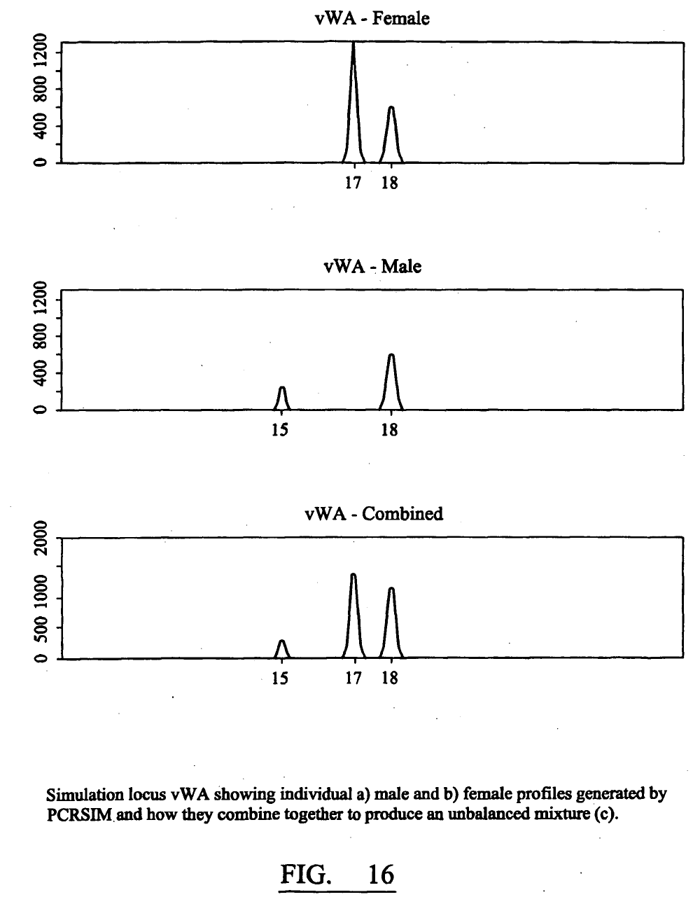

FIG. 14 is a graphical model describing the process according to an embodiment of the invention, for diploid cells;

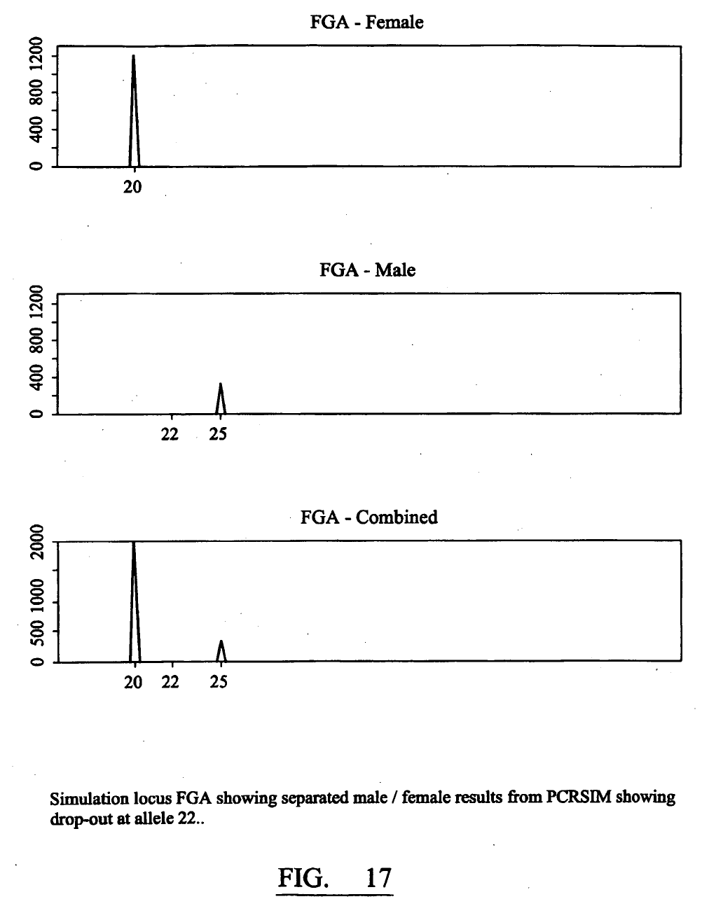

FIG. 15 as a simulation of SGM plus LCN-STR profiles from a mixture of 50 female cells and 20 male cells. PCR amplified 34 cycles—counts of the y-axis were standardised by 2.35×10⁷ (T) and then scaled by 2×10⁶-stutter module was not used in this simulation;

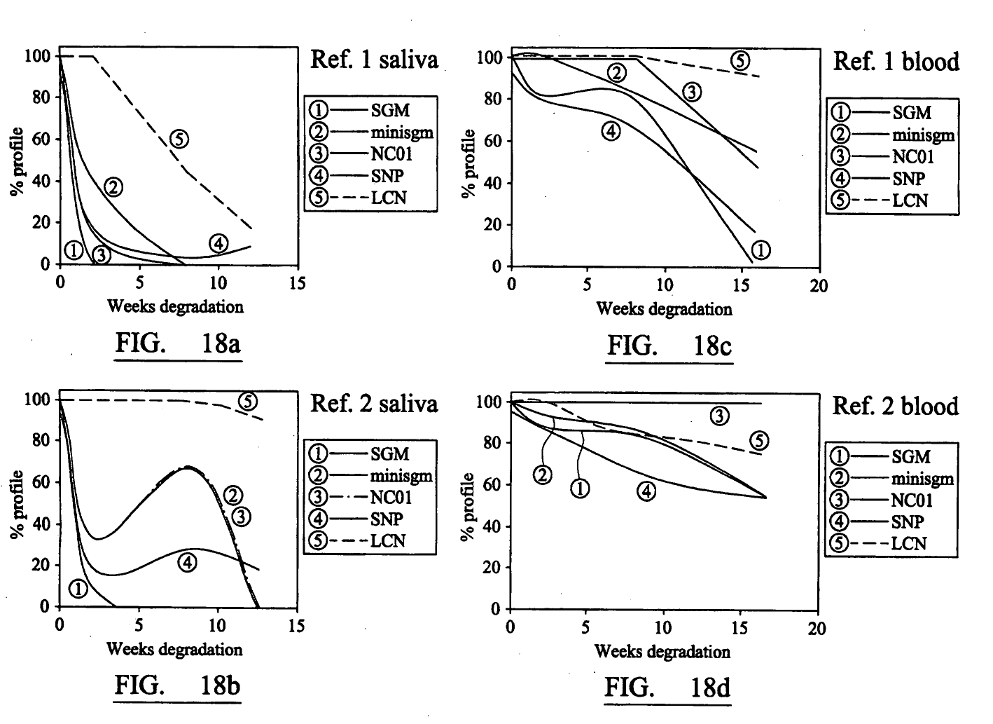

FIG. 16 is a simulated locus vWA showing individual a) male and b) female profiles generated by the invention and how they combine together to produce an unbalanced mixture (c);

FIG. 17 is a simulated locus FGA showing separated male/female results from the invention showing drop-out at allele 22;

FIG. 18a illustrates the effect of degradation on the completeness of profile obtained with respect to a number of analysis techniques for a first saliva sample;

FIG. 18b illustrates the effect of degradation on the completeness of profile obtained with respect to a number of analysis techniques for a second saliva sample;

FIG. 18c illustrates the effect of degradation on the completeness of profile obtained with respect to a number of analysis techniques for a first blood sample;

FIG. 18d illustrates the effect of degradation on the completeness of profile obtained with respect to a number of analysis techniques for a second blood sample;

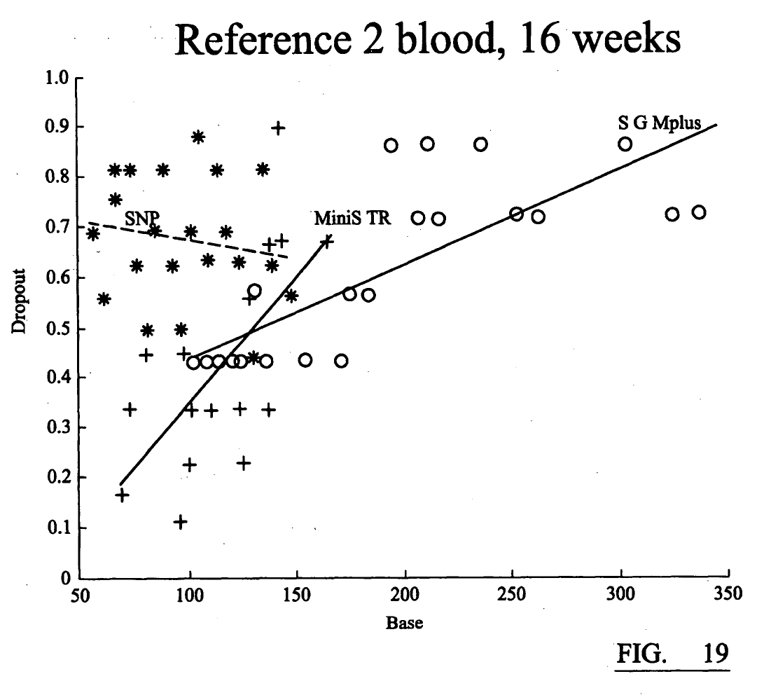

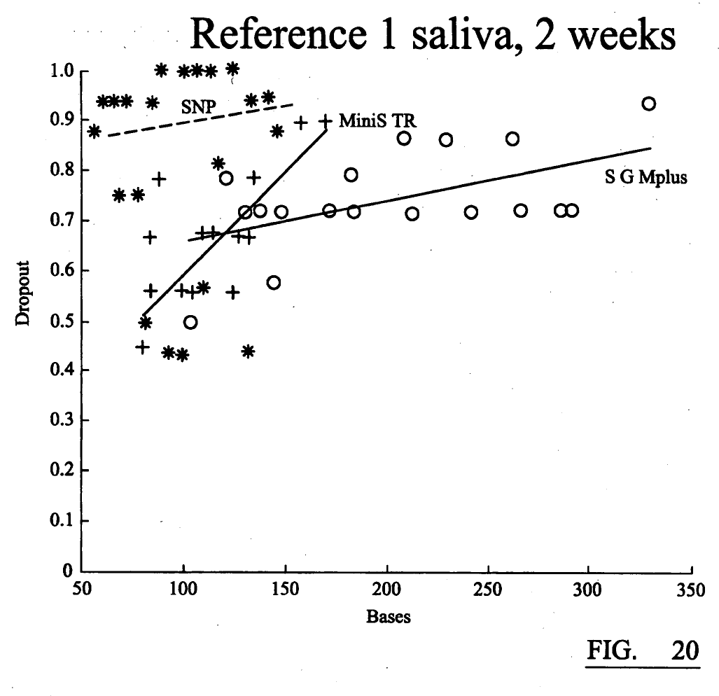

FIG. 19 illustrates the extent of drop out with respect to fragment base size using SNP, mini-STR and STR based analysis for the second blood sample after 16 weeks;

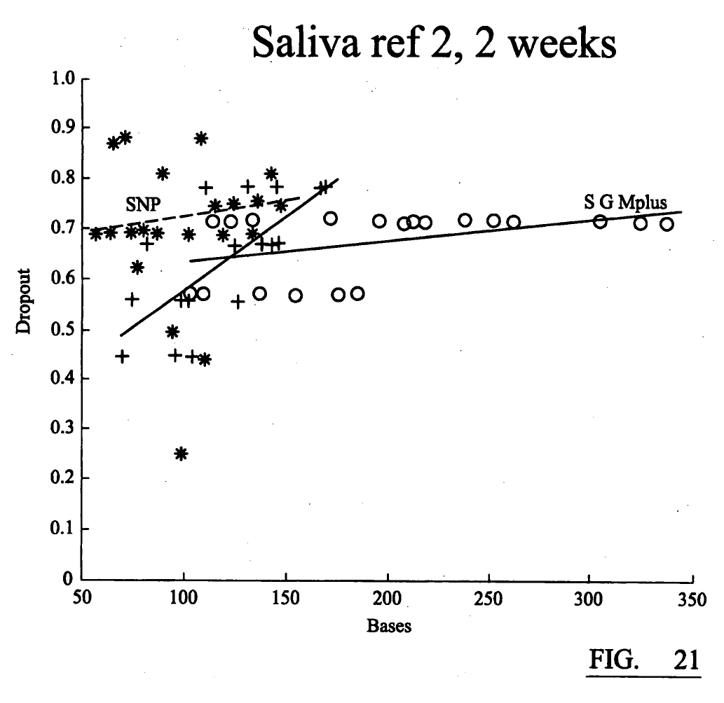

FIG. 20 illustrates the extent of drop out with respect to fragment base size using SNP, mini-STR and STR based analysis for the first saliva sample after 2 weeks;

FIG. 21 illustrates the extent of drop out with respect to fragment base size using SNP, mini-STR and STR based analysis for the second saliva sample after 2 weeks;

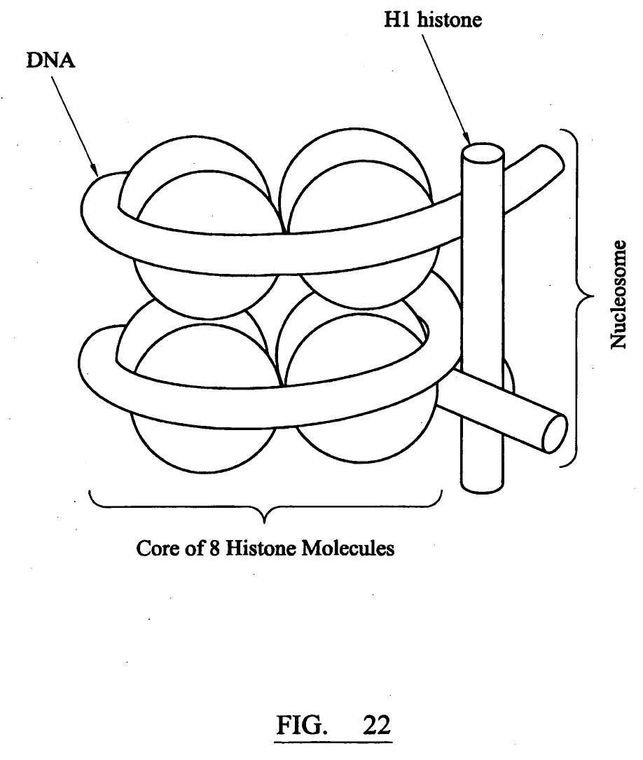

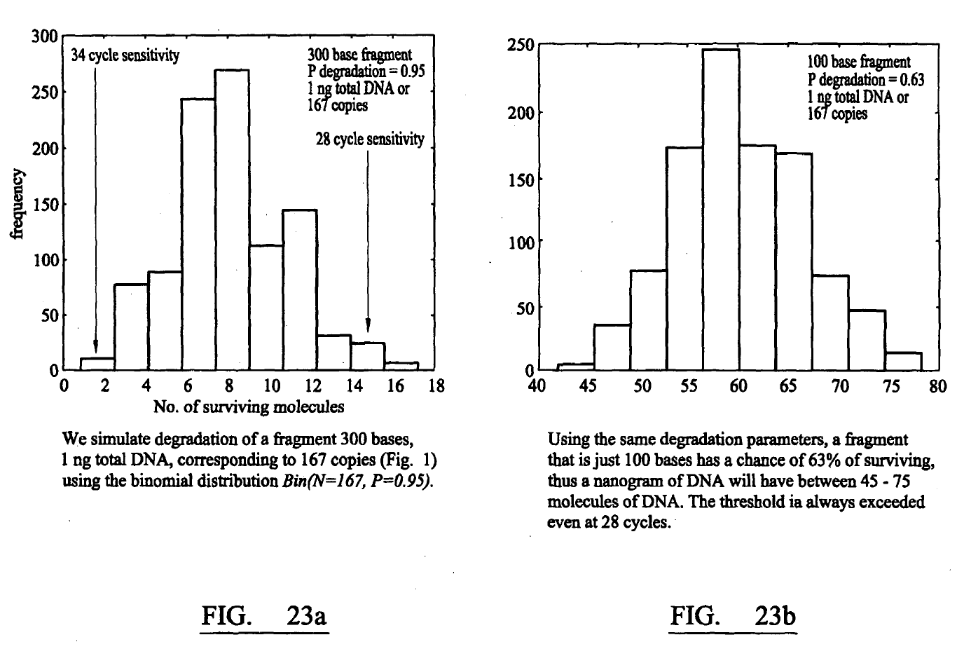

FIG. 22 illustrates the structure of a nucleosome;

FIG. 23a illustrates the frequency against number of surviving molecules plot for a 300 base fragment;

FIG. 23b illustrates the frequency against number of surviving molecules plot for a 100 base fragment; and

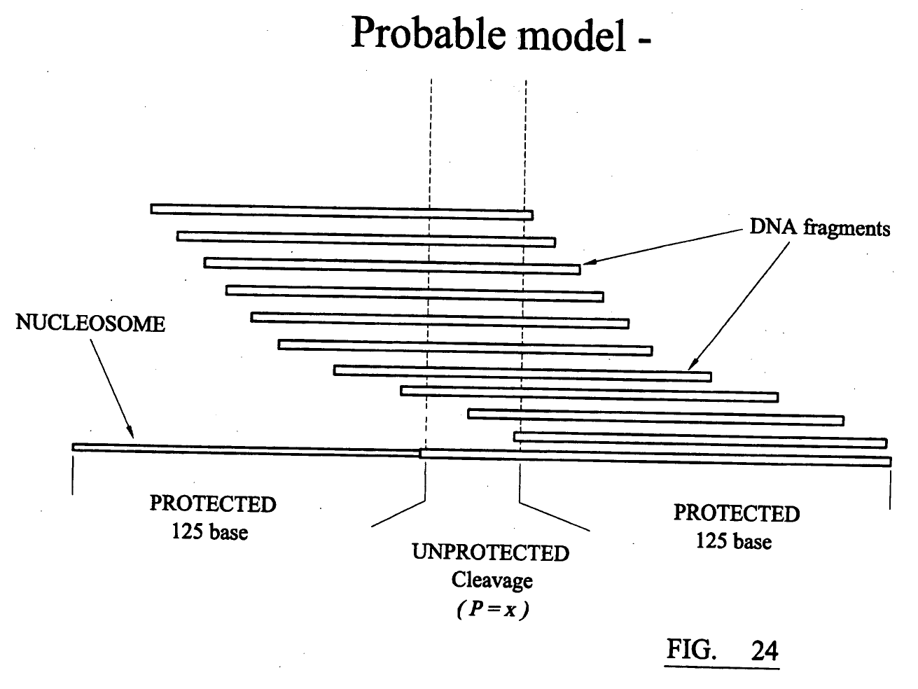

FIG. 24 illustrates a potential model for protect and unprotected DNA with respect to degradation.

### BACKGROUND

In many situations there is a need to consider the DNA present in a sample so as to provide useful information. Within this range of situations, various different issues which impact upon the ability of the DNA consideration process to provide that information exist.

For example, in forensic, ancient DNA and some medical diagnostic applications there may be only limited, highly degraded DNA available (<100 pg) for analysis. To maximise the chance of a result, sufficient PCR cycles must be used to ensure that at least a single template molecule will be visualised.

When short tandem repeat (STR) DNA is analysed, there are 2 main problems that result from stochastic events: one or more alleles of a heterozygous individual may be completely absent—this is known as allele drop-out—Gill, P., J Whitaker, et al. (2000). “An investigation of the rigor of interpretation rules for STRs derived from less than 100 pg of DNA.” Forensic Sci Int 112(1): 17-40.; and/or PCR generated slippage mutations or stutters—Walsh, P. S., N. J Fildes, et al. (1996). “Sequence analysis and characterization of stutter products at the tetranucleotide repeat locus vWA.” Nucleic Acids Res 24(14): 2807-12. may be generated. Both events may compromise interpretation.

In relation to these issues, and other issues peculiar to forensic applications (the sample itself may be a mixture of 2 or more individuals) attempts have been made to detail the principles involved and improve particular steps in the generation of the results and/or the interpretation of the results. These efforts have concentrated on individual steps of the process and have generally been concerned only with the PCR steps in the process. For instance, mathematical models to describe STR mutation slippage or stutter mutations during PCR have been developed: Sun, F. (1995). “The polymerase chain reaction and branching processes.” J Comput Biol 2(1): 63-86.; Lai, Y. and F. Sun (2004). “Sampling distribution for microsatellites amplified by PCR: mean field approximation and its applications to genotyping.” J Theor Biol 228(2): 185-94.; Shinde, D., Y Lai, et al. (2003). “Taq DNA polymerase slippage mutation rates measured by PCR and quasi-likelihood analysis: (CA/GT)n and (A/T)n microsatellites.” Nucleic Acids Res 31(3): 974-80. However, these only simulate a part of the PCR process, use a totally different probability theory (random binary trees) to describe probabilistic relationships and are concerned with dimeric microsatellites which are inherently difficult to interpret as PCR slippage mutations occur at relatively high frequency at these loci.

### OVERVIEW OF THE INVENTION

The present invention provides for the first time a simulation of the complete DNA consideration process. As illustrated in FIG. 1, the simulation takes the DNA consideration process through from the start to the end. The simulation goes through all the stages: extraction→aliquot into pre-PCR reaction mix→PCR amplification for t cycles→visualisation of alleles after electrophoresis.

The above described basic simulation can be supplemented using simulations of other steps and/or issues. For instance, it is possible to simulate the expected variation in PCR stutter artefact, heterozygote balance, and to predict drop-out rates.

By providing such a simulation, the present invention contributes greatly to the understanding of the dependencies of parameters associated with the DNA consideration process. Such a computer model based simulation also allows a variety of other benefits to be obtained and new approaches to the DNA consideration process to be taken.

As will be explained in greater detail below, the invention preferably uses: experimental data to predict input parameters for various steps in the process; binomial functions of the form Bin (n, π) to simulate all the steps (where n is the number of template molecules and π is an efficiency parameter between 0-1); and a graphical model or Bayes net solution to combine the steps. In particular, the invention uses inputs to the simulation consisting of N cells; extracted with π_{extraction} efficiency; an aliquot of ×ul (π_{aliquot}) is removed from the extract; this is added to the pre-PCR reaction mix; then t cycles of PCR amplification are carried out with π_{PCReff} efficiency

No description of the entire DNA consideration process by computer simulation has been provided before. To do this, the applicant has first simulated each part of the DNA consideration process, and then used a graphical model or Bayes net solution to combine the parts. Each part of the process is represented by a node in the graphical model—each node comprises parameters and a distribution and is dependent upon other nodes in the model. Modelling processes in this way is intuitive and simplifies the complex inter-dependencies that are inherent in the multiple stochastic effects that are prevalent in the process of DNA analysis.

Furthermore, the applicant then demonstrates below that such models can be used to assess and measure unknown variables such as extraction rate, or to optimise parameters such as the amount of pe-PCR aliquot taken. By modelling ‘what-if’ scenarios, the invention allows the entire DNA consideration process or steps therein to be improved, and this translates into improved success rates when real samples are analysed.

### DETAILED VIEW OF THE PRESENT INVENTION

Details on the approach taken in the present invention are now provided in a number of sections. These give details on:
i) the approach taken to obtain the experimental data, including that used to predict input parameters for various steps in the process;
ii) details of the input and output parameters considered;
iii) explanation of the form of the binomial functions used to simulate all the steps and justification for the applicability thereof;
iv) details of how a graphical model or Bayes net solution is used to combine the steps.

### Experimental Data

Materials and methods

### DNA Extraction and Quantification

DNA was extracted using Qiagen™ QiaAmp Mini-Kits (Cat. No. 51306) or Qiagen™ Genomic-Tip system (Cat no. 10223, 20/G tips). Samples had been stored frozen at −20° C. and were defrosted at room temperature prior to DNA extraction. The manufacturers' protocol for each sample type was used to obtain between 0-2 ng/μL DNA (Mini-Kits) or 5-15 ng/μL DNA (Genomic-Tips), suspended in 1× TE Buffer (ABD). Samples were quantified using Picogreen and/or the Biochrom UV spectrophotometer Hopwood, A., N. Oldroyd, et al. (1997). “Rapid quantification of DNA samples extracted from buccal scrapes prior to DNA profiling.” Biotechniques 23(1): 18-20. We also carried out real time PCR quantification using the Applied Biosystems (Foster City, Calif., USA) Quantifiler Human Kit™ and Quantifiler Y Kit™ Taq man assays, following the manufacturer's protocol ref) (http://docs.appliedbiosystems.com/pebiodocs/04344790.pdf).

### SGM Plus™ PCR Amplification

The method of Cotton, E. A., R. F. Allsop, et al. (2000). “Validation of the AmpflSTR SGM plus system for use in forensic casework” Forens. Sci. Int. 112: 151-161. was followed: AMPFISTR® SGMplus™ kit (Applied Biosystems, Foster City, Calif., USA) containing reaction mix, primer mix (for components see Perkin Elmer user manual), AmpliTaq Gold® DNA polymerase at 5 U/μl and AMPFISTR® control DNA, heterozygous for all loci in 0.05% sodium azide and buffer was used for amplification of STR loci. DNA extract was amplified in a total reaction volume of 50 μl without mineral oil on a 9600 thermal cycler (Applied Biosystems GeneAmp PCR system) using the following conditions: 95° C. for 11 minutes, 28 cycles (or 34 cycles for LCN amplification) of 94° C./60 s, 59° C./60 s, 72° C./60 s; 60° C. extension for 45 minutes; holding at 4° C.

Sample data from the 377 instrument was analysed using ABI Prism™ Genescan™ Analysis v3.7.1 and ABI Prism™ Genotyper™ software v3.7 NT. Data extracted from Genotyper™ (peak height, peak area, scan number, size in bases).

### Laser Micro-Dissection (LMD)

The method of Elliott, K, D. S. Hill, et al. (2003). “Use of laser microdissection greatly improves the recovery of DNA from sperm on microscope slides.” Forensic Sci Int 137(1): 28-36. was used to select N sperm or epithelial cells from microscope slides.

### Case Work Analysis Approach

The current casework analysis approach, using the second generation multiplex (SGM-plus) system Cotton, E. A., R. F. Allsop, et al. (2000). “Validation of the AmpflSTR SGM plus system for use in forensic casework” Forens. Sci. Int. 112: 151-161. Martin, P. D. (2004). “National DNA databases—practice and practicability. A forum for discussion.” Progr. Forens. Genet. 10: 1-8. was mirrored in the present invention. This case work analysis approach is currently used in all casework in the UK.

### Sample Purification

Samples are typically purified using Qiagen columns (QIAamp DNA minikit; Qiagen, Hilden, Germany) (ref). A small aliquot (2 ul) of the purified DNA extract is then quantified using a method such as picogreen assay; then a portion is removed to carry out PCR. Dependent upon the casework assessment, coupled with information about the quantity of DNA present, a decision is made at that point whether to analyse using 28 cycles (conventional >250 pg in the total PCR reaction) or whether LCN protocols are followed Gill, P., J. Whitaker, et al. (2000). “An investigation of the rigor of interpretation rules for STRs derived from less than 100 pg of DNA.” Forensic Sci Int 112(1): 17-40., using 34 PCR cycles, if less than 250 pg and/or the DNA is highly degraded. After PCR, the samples are electrophoresed using AB 377 instrumentation. Genotyping is automated using Genescan, and Genotyper software. Allele designation is carried out with the help of expert systems “STRESS” Werrett, D. J., R. Pinchin, et al. (1998). “Problem solving: DNA data acquisition and analysis.” Profiles in DNA 2: 3-6. and “True Allele” (Cybergenetics, Pittsburgh, USA, http://www.cybgen.com/. If mixtures are present then an expert system PENDULUM, Gill, P., R. Sparkes, et al. (1998). “Interpreting simple STR mixtures using allele peak areas.” Forensic Sci Int 91(1): 41-53. is used to devolve genotype combinations.

### Input and Output Parameters

The invention provides a MATLAB based simulation program (rewritten into C++) that exactly follows the DNA extraction process at the molecular level. The process can be defined by a series of input and output parameters as follows:

### Input Parameters:

1) No. cells (N): typically a stain or sample will contain N cells. Each diploid DNA cell comprises c. 6 pg of DNA and a haploid cell comprises 3 pg DNA. Given a DNA concentration, it is possible to convert this into an equivalent number of haploid or diploid cells
2) Extraction efficiency (π_{extraction}): During the process of extraction, the cells are disrupted and the DNA liberated into solution. During extraction there is a probability π_{extraction} (the extraction efficiency) that a given DNA molecule will survive the process and be present in the extracted sample.
3) Aliquot (π_{aliquot}): A portion only of the extracted sample is submitted for PCR. Therefore, there is a finite probability π_{aliquot} that a given molecule will be selected from all of those in the extracted sample and so be present in the portion subjected to PCR.
4) PCR efficiency (π_{PCReff}): PCR is not 100% efficient; hence during each round there will be a finite probability π_{PCReff} that a DNA fragment will be amplified.
5) No. of PCR cycles (t_{cycles}): Typically t=28 for normal DNA profiling and t=34 cycles for LCN, but the number of cycles effects the extent and form of amplification.

### Output Parameters:

1) Probability of allele drop-out, p(D): The chance that an allele will fail to amplify.
2) Number of amplified molecules (n_{A}, n_{B}): The simulated number of molecules for a given allele A or B can be measured and compared against threshold level T that must be achieved in order for a signal to be observed (this is approximately 10⁶/ul of PCR amplification product). Note for 34 cycle PCR T is always achieved
3) Heterozygote balance (Hb): For a given heterozygote locus we derive a distribution of Hb=n_{A}(t)/n_{B}(t).

### The Form of the Binomial Functions

### Input Parameters

### 1) Number of Cells

The general approach of the present invention allows a wide variety of values of n, and the implications thereof, to be considered. For instance, high n values may result in too much DNA after PCR and hence problems in analysis. At the other end of the scale, an important issue is the minimum number of cells which are needed for the DNA in the sample to be accurately reflected in the analysed DNA sample. The binomial approach can be used for all these questions, including in respect of both haploid and diploid cells.

In doing so, the invention takes into account that for a given heterozygote it is not valid to assume that equivalent numbers of both alleles are present before PCR. Additionally, the provision of a formal statistical model simplifies the approach.

The difference between haploid (sperm) and diploid cells needs to be noted, however. Whereas a single diploid cell has each allele at a locus represented once (i.e. in equal proportions) this is not true for haploid cells. For example, if only one haploid cell is selected then just one allele can be visualised. The chance of selecting alleles A or B at a locus is directly dependent upon the number of sperm analysed. We can assess the chance of simultaneously observing alleles A and B using the approach below.

To calculate the chance of observing alleles A and B in a sample of n sperm at a heterozygous locus, the consideration in FIG. 2, the probability of observing at least one copy of allele A and at least one copy of allele B is calculated. This satisfies the four conditions necessary for a Binomial model, namely.
a) The number of trials (or sample size) must be fixed in advance—in this case, n sperm;
b) There are only two possible outcomes for any trial—in this case, either allele A or allele B;
c) The trials are independent—in this case, the probability that any given sperm has allele A or allele B is independent of the probability of any other sperm having A or B;
d) The probability of success is constant—in this case, Pr(i th sperm is A)=p_{A} i=1 . . . n.

Therefore, if we define Pr(A=x & B=y) to be the joint probability that there are observed x copies of allele A and y copies of B then:

$$\begin{matrix}Pr ( A \geq 1 \& B \geq 1 ) = 1 - \overline{Pr ( A \geq 1 \& B \geq 1 )} \\ = 1 - Pr ( A = 0 or B = 0 ) \\ = 1 - [ \begin{matrix}Pr ( A = 0 ) + Pr ( B = 0 ) - \\ Pr ( A = 0 \& B = 0 )\end{matrix} ] \\ = 1 - [ {( 1 - {p}_{A} )}^{n} + {p}_{A}^{n} ]\end{matrix}$$

And if p_{A}=0.5=(1−p_{A})=p_{B} then this becomes 1−0.5ⁿ⁻¹

So the question alternative question of how many sperm (n) are needed to be 100 p % confident that both alleles are observed (if the person is truly heterozygous) is given by:

$$n = 1 + \frac{log ( 1 - p )}{log ( 0.5 )}$$

This will not give integer values, so the recommended number would be the ceiling value of this expression. The result of this consideration is presented graphically in FIG. 2. At least 6 sperm are required to be 95% certain; 8 sperm are needed to be >99% certain. This theoretical limit is the best possible that can be achieved under the assumption that a single allelic template can be detected in an extract. This relationship should work well for direct PCR methods. In practice more sperm are required since extraction methods are inefficient and consequently DNA will be lost prior to PCR.

### 2) Extraction Efficiency

As just mentioned, the efficiency of extraction is another issue which needs to be taken into account. Typically, the Qiagen method of extraction is used. This involves the addition of chaotropic salts to an extract of a body fluid and subsequent purification using a silica column. At the end of the process, purified DNA is recovered. Unfortunately some of the DNA is lost during the process and is therefore unavailable for PCR. The parameter π_{extraction} describes the extraction efficiency. For example, if n target DNA molecules are extracted with π_{extraction}=0.5, then approximately n/2 molecules are recovered in the step. The general approach of the present invention allows variation in this respect to be accommodated and its effect considered.

Once again, the extraction process is simulated using the binomial approach, r=Bin(2N, π_{extraction}) where r is a random number from the binomial distribution. On this basis, 1000 samples can be considered to form a distribution, and with N as the number of diploid cells and (in this example) with π_{extraction}=0.6 then the results of FIG. 3 are obtained. For example, if 10 cells are extracted, then between 5 and 18 copies of DNA template per locus will be recovered.

### 3) Aliquot Size/Proportion

In practice an aliquot will be forwarded for PCR amplification—this enables repeat analysis if required. Typically, out of a total extract of 66 ul, a portion of 20 ul will forwarded for PCR. The selection of template molecules by pipetting can also be modelled using another binomial distribution of the form, Bin(n, π_{aliquot}), where π_{aliquot}=20/66 ul (the aliquot proportion). The 20 ul extract is then forwarded into a PCR reaction mix to make a total 50 ul.

FIG. 4 shows probability density functions simulating template recovery (n) from 5, 10 and 20 diploid cells when 20/66 ul aliquots are taken from an extract. A comparison of FIGS. 3 and 4 demonstrates that using this technique at least 20 cells are needed to avoid allele drop-out. If 5 cells are extracted then 35% of heterozygous loci will exhibit allele drop-out. The crucial values from the various steps are thus identified and can be considered.

### 4) PCR Efficiency and 5) Number of PCR Cycles

PCR does not occur with 100% efficiency. The amplification efficiency (π_{PCReff}) can range between 0-1. The process can be described by nₜ=n₀(1+π_{PCReff})^{t} Arezi, W. Xing, et al. (2003). “Amplification efficiency of thermostable DNA polymerases.” Anal Biochem 321(2): 226-35. where nₜ is the number of amplified molecules, n₀ is the initial input number of molecules and t is the number of amplification cycles. However, a strictly deterministic function will not model the errors in the system, especially if we are interested in low copy number estimations (e.g. less than 20 target copies).

Again the modeling of the PCR amplification in the present invention uses the binomial function. The first round PCR replicates the available template molecules per locus (n₀) with efficiency π_{PCReff} to produce n₁ new molecules per locus:

n₁=n₀+Bin(n₀,π_{PCReff})

For the second round of PCR both n₀ and n₁ are available hence:

n₂=n₀+n₁Bin(n₀+n₁,π_{PCReff})

If there are t PCR cycles then it can be generalized that the final number of molecules generated per locus is:

$${n}_{t} = {\sum }_{i = 1}^{t - 1} + Bin ( {n}_{t - 1} , {π}_{PCReff} )$$

### Output Parameters

### 1) Probability of Allele Dropout

By simulating nₜ 1000 times it is possible to estimate the variation. For low copy number typing there are typically t=34 PCR cycles. We have empirically demonstrated that this is sensitive enough so that a single target copy will be visualized because it will always produce sufficient molecules to exceed the detection threshold (T) i.e. >2×10⁷ molecules in the total of 50 ul PCR reaction, FIG. 5, or c. 4×10⁵ per ul of amplified DNA. We can generalize that for 34 cycle PCR, that the phenomenon of drop-out is dominated solely by the absence of template in the pre-PCR mix—predicted levels of dropout pre- and post-PCR are the same in FIGS. 4 and 5. However, if the number of PCR cycles is reduced to a level that does not produce sufficient copies to trigger the threshold level (T) then there will be a failure to detect, FIG. 6, i.e. p(D) is comprised of 2 components:

p(D)=p(D)+p(DT)

where p(Dₛ) is the pre-PCR stochastic element and p(DT)=p(nₜ<T).

In the context of this simulation, it is possible to provide an experimental estimation of PCR efficiency—π_{PCReff}

Through real time PCR, using a commercial Applied Biosystems Y-Quantifiler kit (refs 20), it is possible to estimate the quantities of DNA present. This method employs a 70 base Y chromosome fragment that is PCR amplified in real-time. A series of C_{T} values were calculated for 23-50,000 target copies (data not shown). From the regression of the C_{T} slope we estimated π_{PCReff}=10^{[-1/slope]}−1 (Ariz et al) and determined π_{PCReff}=0.82+−0.12 SE.

This estimate also corresponded well when we iterated π_{PCReff} to minimise (observed−expected)² residuals from Hb output when known quantities of DNA were PCR amplified (data not shown). Throughout, we have used π_{PCReff}=0.8.

### 2) Number of Amplified Molecules—Quantification

Quantification is carried out after DNA extraction and purification with the purpose of ensuring that there are sufficient DNA molecules (n₀) in the PCR reaction mix, so that after t amplification cycles nₜ molecules are produced. The aim is to ensure that nₜ>T. If nₜ<T then allele drop-out will occur because the signal is insufficient to be detected by the photomultiplier. A number of different methods can be utilised e.g pico-green assay Hopwood, A., N. Oldroyd, et al. (1997). “Rapid quantification of DNA samples extracted from buccal scrapes prior to DNA profiling.” Biotechniques 23(1): 18-20. to allow physical quantification.

Generally, when levels of DNA are <0.05 ng/ul, then results tend to be unreliable Kline M C, Duewer, D L, Redman J W, Butler J M (2004) Results from the NIST 2004 quantitation study—in press J Forensic Sci. However, newer methods based on real time Taq man assays (e.g. AB Quantifiler kit) Richard, M L., R. H. Frappier, et al. (2003). “Developmental validation of a real-time quantitative PCR assay for automated quantification of human DNA.” J Forensic Sci 48(5): 1041-6. appear to offer much higher sensitivity and will in turn make the decision making process more reliable. Alternatively, if too much DNA is applied then the electrophoretic system will be overloaded. Generally, multiplexed systems are optimised to analyse c. 250 pg-1ng DNA. Hence, in practice the quantification process is used to decide π_{Aliquot} discussed above, and which is therefore an operator dependent variable. Generally this ranges from 1-20 ul and is used to optimise n₀. The number of PCR cycles (t) is also a variable (either 28 or 34 cycles in most examples used by the applicant) and this decision is also dependent upon an estimate of n₀.

Quantification estimates the quantity (pg) of post-extracted DNA in a sample. There are approximately 6 pg per cell nucleus, hence we can estimate the equivalent number of (2n) target molecules that are input into the simulation model at the PCR stage.

### 3) Heterozygote Balance

The present inventions approach to simulation is also applicable to the consideration of the ratio of one allele A to the other B in the amplified product.

For a heterozygote locus with alleles A and B, for each allele the number of post-PCR molecules n_{A} (t) was simulated 1000 times. Given the 2 parameters π_{Aliquot} and π_{PCReff} 1000 estimates were obtained of Hb=min (n_{A}(t), n_{B}(t))/max (n_{A}(t), n_{B}(t))

Simulation results were compared to experimental data from 1692 samples where c. 1 ng of DNA was analysed. A best fit was achieved by iterating n and it was found that experimental data corresponded to a best fit of c. 500 pg DNA input into the pre-PCR reaction mix. This is c. 83 diploid cells. If more cells were input, then the simulation produced unrealistically high heterozygous balance (data not shown), hence we concluded that at >500 pg template, the PCR reaction ceased to be log-linear, reaching a plateau phase before the final cycle has been reached (t=28). Whereas this could be modelled more effectively, the greater interest is in low copy number DNA template situations (t=34) where stochastic effects are marked.

A choice of a single parameter for π_{PCReff}=0.8 was shown to work well for all simulations. Provided that sufficient template was produced to trigger the threshold level T then the model was not very sensitive to changes in π_{PCReff}. FIG. 7 demonstrated that there was very good agreement between the simulation and observed results. The results also confirm a strong theoretical basis for the widespread utilised parameter guideline that defines Hb>=0.6, Gill, P., R. Sparkes, et al. (1997). “Development of guidelines to designate alleles using an STR multiplex system.” Forens. Sci. Int. 89: 185-197., which is used to assist interpretation of mixtures when optimal amounts of DNA are analysed.

In the context of LCN situations, the impact of a 25 pg pre-PCR input was simulated and gave the results of FIG. 8.

In this case, Hb becomes much more variable, although drop-out was not encountered. This also illustrated the importance of maximizing n₀ in the pre-PCR reaction—in previous experiments significant dropout was encountered when 5 cells were diluted into 20/66 ul. Once again the simulation and experimental data gave a very good fit. This time it was not necessary to iterate any of the input parameters, since at lower levels of DNA, the PCR amplification stayed in the log-linear phase throughout.

Modelling of more complex scenarios enables estimates of parameters such as π_{extraction}. Laser micro-dissection was used to select 10 epithelial cells and these were purified by Qiagen columns, with a π_{Aliquot}=20/66, and a t=34 PCR cycles. Simulation proceeded by iterating π_{extraction} revealed that the simulation was relatively insensitive to π_{PCReff} and that provided that nₜ>T, then p(D) was independent of π_{PCReff}. Iterating π_{extraction} also showed that the residuals of Hb minimise when π_{extraction}=0.46. In addition, the p(D) residual is simultaneously minimised, thus establishing p(Hb) and p(D) are dependent—the latter is an extreme consequence of the former. There is quite a high loss of DNA during extraction in this example, and demonstrates that the lower the amount of DNA that is purified, the less that can proportionately recovered by the Qiagen extraction methods. The results are provided in FIG. 9.

In an experiment which considered 1-55 sperm cells (N) from an individual of known genotype, analysed as described previously, then a plot of N v. observed p(D) demonstrated a log₁₀ linear relationship. Iterating against π_{extraction} the best fit is 0.3, FIG. 10. The comparisons, are very close i.e. residuals are small, which indicated that the model was robust. At a practical level, it appeared that the success rate for extracting sperm was much less than for epithelial cells, c.f. FIG. 9.

As a result of the above, a demonstration has been provided that the invention's simulation is adequate to describe the key output parameters of STR analysis, namely heterozygous balance and allele dropout.

### Extension of the Simulation

One of the significant advantages of the simulation and this approach to it is that the simulation of steps can be inserted or removed and yet the underlying concept still be beneficial. Thus one or more steps of the simulation above can be omitted. Equally, it is possible to include in the simulation other steps and issues. One such issue is stutter, and this is discussed next, with the issue of degradation discussed later.

Stutters are artefactual bands that are produced by molecular slippage of the Taq polymerase enzyme. This causes an allelic band to alter its state from its parent, in vivo, state during successive amplifications. The presence of stutter may compromise the interpretation of some mixtures especially where there are contributions from 2 individuals in a ratio <c. 2:5 because the minor allelic components can be the same peak area size as stutters from major contributor. Therefore, it is important to model.

The invention thus assesses πₛₜᵤₜₜₛ the chance that Taq enzyme slippage leads to a stutter. This can happen only during PCR, hence the number of stutter templates in the pre-PCR (n₀) reaction mix is always zero. πₛₜᵤₜₜ is approximately 400 times less than π_{PCReff}.

Once a stutter is formed, then it acts as template identical to a normal allele (as the sequence is the same as an allele 1 repeat less than the parent). Consequently the propagation of stutter is exponential with efficiency π_{PCReff} and after t cycles forms n_{S} stutter molecules. In the electropherogram, the quantity of stutter band is always measured relative to the parent allele:

$$p ( {S}_{A} ) = \frac{φ {S}_{A}}{φ A}$$

where φ=peak area or peak height of the stutter (S_{A}) and allele (A) respectively.

In practice, c. 5% alleles fail to produce visible stutter ie n_{S}<T.

The relative peak area of stutter is variable between loci and also between alleles Shinde, D., Y. Lai, et al. (2003). “Taq DNA polymerase slippage mutation rates measured by PCR and quasi-likelihood analysis: (CA/GT)n and (A/T)n microsatellites.” Nucleic Acids Res 31(3): 974-80., therefore it may appropriate to evaluate stutter at every allelic position. In order to assess this, locus D3 from the SGM plus system was chosen and probability density functions (pdfs) of stutter peak areas were prepared:
a) Across all stutters regardless of whether the parent allele was homozygous or heterozygous;
b) When stutters were only associated with heterozygotes;
c) When stutters were associated with parent allele 15 only, i.e at the allele 14 position.

Comparison showed there was little difference between the density estimates, FIG. 11, although the subset of data that related specifically to allele 15 gave the most discrete distribution.

Based on all D3 observations, π_{Stutter} was modelled with Beta distribution. The parameters of the Beta distribution where chosen so that, the distribution of π_{Stutter} had a mean of μ_{π}_{_{Stutter}}=0.002 and a variance of σ_{π}_{_{Stutter}}²=2.25×10⁻⁶. This can be done by using the following identities:

If X˜Beta(α, β), and E[X]=μ_{X}, sd(X)=σ_{X}, then

$$α = {μ}_{X} ( \frac{{μ}_{X} ( 1 - {μ}_{x} )}{{σ}_{X}^{2}} - 1 )$$

$$and$$

$$β = ( 1 - {μ}_{X} ) ( \frac{{μ}_{X} ( 1 - {μ}_{x} )}{{σ}_{X}^{2}} - 1 ) .$$

For the given mean and variance this results in α=1.77 and d β=884.34 (FIG. 12). The minimised residuals were achieved when π_{Stutter}=0.002, a figure reached by estimation.

In the case of degradation, the passage of time results in the breakdown of DNA. The greater the time that passes the smaller the fragments that are left become. Eventually this means that breaks occur within the fragment length being considered to establish a SNP or STR identity and so that particular fragment is not available for amplification and consideration. If this occurs for a large number of the instances of a fragment then it may in effect drop out of the detected result. Such drop out can be additional to or instead of the drop out caused by stochastic effects, particularly in small DNA samples.

As with the other issues, it is possible to account for degradation within the model. As part of the investigations to do so, two blood sample and two saliva samples were taken, split into a large number of aliquots and then degraded to varying degrees before analysis. Degradation was achieved by incubating the aliquots in humid tubes for a variety of times between two and sixteen weeks. Multiple analyses of the aliquots were then perform using various analysis techniques including SGMplus, min-SGM, NC01, and SNP-plex. The aliquots were also examined by Low Copy Number analysis, LCN. The results for the first saliva sample are set out in FIG. 18a, for the second saliva sample in FIG. 18b, for the first blood sample in FIG. 18c and for the second blood sample in FIG. 18d.

Generally speaking the increased cycles and other steps taken in LCN is successful in obtaining a fuller profile for longer.

The information on the impact of degradation with time from these investigations assists in forming the model. This needs to account for the degradation of DNA which tends to degrade due to the action of DNAase and/or non-specific nuclease, the latter cleaving any base.

On the basis that any base has an equal probability of cleavage then (from cumulative binomial distribution):

P_{fragment}=1−(1−p_{deg/base})^{bases}

could be used to model the impact of this issue. Thus the chance that a fragment will degrade/decompose is dependant upon a degradation parameter, p_{deg/base}, which is the chance that a single base will cleave. This could be treated as constant for all bases, but, again investigations have been used to inform on the process.

FIG. 19 presents a plot of drop out (increasing up the y axis) against the size of the target fragment size expressed in terms of bases. Targets considered using SNP, mini STR and STR based approaches were considered. In this case all of the results relate to the second blood sample after 16 weeks. FIG. 20 represents an equivalent plot for the first saliva sample after 2 weeks and FIG. 21 represents an equivalent plot for the second saliva sample after 2 weeks. In each figure the *results are SNP results, the + results are mini-STR results and the O results are STR results. Applied to each of the sets of data is a regression. In each case there is a crossing of the regressions, a point of inflexion, at around 125 bases. The investigations have thus shown that the value of p_{deg/base} is size specific and hence is specific to the particular fragment/target being considered.

By way of explanation of this, DNA is condensed and wrapped around histone proteins called nucleosomes, as shown in FIG. 22. There are four histone proteins (H2A, H2B, H3, H4) with two copies of each forming an octomer core. The length of the complete turn of the DNA helix around the histone molecules is 146 bases. As such, it would appear that a large part of this DNA is protected from degradation by the complex and only the expose areas of DNA are at risk from cleavage, particularly by DNAase. Hence degradation proceeds preferentially in respect of DNA fragments greater than the protected size, with the investigation pointing to a size around 125 bases as being the protected length. This suggests that approximately 10 bases at either end of each turn of the helix are exposed to degradation.

Because of this effect, the degradation parameter, P_{deg/base}, is best treated as potentially different for each fragment/target, and so take into account the fragment/target size too.

Using such a model, and assuming a 95% chance that any given base will cleave after degradation has reached the model stage, then it is possible to simulate the results for 1 ng of DNA (167 copies) for fragment sizes of 300 bases and 100 bases respectively. The general expectation is that the issue of degradation is more significant for the larger fragment size as there is less chance of such fragment length surviving cleavage, however, the simulation of the present invention allows far more detail to be determined. Referring to FIG. 23a the results for the 300 base fragment size are presented in terms of a plot of frequency against the number of surviving molecules. In FIG. 23b an equivalent plot is provided for the 100 base fragment. For the 300 base fragment with Bin (N=167, P=0.95) then 28 cycles of amplification is extremely unlikely to produce detectable results compared with the detection threshold. 34 cycles, the LCN approach, is sufficient, however. For the 100 base fragment, there is a 63% chance of any fragment surviving degradation and so the detection threshold with both 28 cycles and 34 cycles is readily exceeded. FIG. 24 provides a schematic illustration of the protected, unprotected, protected sequence for DNA and the potential sites which are susceptible to cleavage occurring for a number of example fragments of interest, randomly distributed with respect to the protected and unprotected parts.

This approach can be extended further to provide still more complicated, but potentially more accurate models of the degradation process. Thus it would be possible to allocated a first probability of cleavage to a first length of the sequence and a second probability for the next part, before returning to the first probability for the next part and so on in a repeat pattern. Thus P_{fragmentA}=1−(1−p_{degA/base})^{bases} for the first 125 bases, before becoming P_{fragmentB}=1−(1−p_{degB/base})^{bases} as for the next X bases, before returning to P_{fragmentA}=1−(1−p_{degA/bases})^{bases} for the next 125 bases, and so on. Another degree of sophistication would come from a first low probability for a length, a medium probability length next to that before a higher probability length is reached, with a transition back through a medium probability to a lower probability again, and so on.

The approach could consider the amount of the profile in each of three categories, to inform on the degradation extent and the importance of considering it. Thus the proportion giving a full profile, the proportion giving a partial profile and the proportion giving no profile could be established. The process could optimise the consideration of the partial or non-profiles, or establish that they can be discounted.

Differences in the extent of degradation between male and female DNA could also be considered.

### Use of a Graphical Model

In the discussion above, the subdivision of the DNA consideration process of FIG. 1 has allowed the steps to be individually characterised by a series of input and output parameters. The discussion has then demonstrated how parameters may be estimated using the approach of the present invention.

To formalise the thinking and to provide a robust framework for the simulation or model it is useful to consider the approach represented as a graphical model or Bayes Net.

The graphical model consists of two major components, nodes (representing variables) and directed edges. A directed edge between two nodes, or variables, represents the direct influence of one variable on the other. To avoid inconsistencies, no sequence of directed edges which return to the starting node are allowed, i.e. the graphical model must be acyclic. Nodes are classified as either constant nodes or stochastic nodes. Constants are fixed by the design of the study: they are always founder nodes (i.e. they do not have parents). Stochastic nodes are variables that are given a distribution. Stochastic nodes may be children or parents (or both). In pictorial representations of the graphical model, constant nodes are depicted as rectangles, stochastic nodes as circles.

FIG. 13 represents a graphical model describing one embodiment of the process for diploid cells. FIG. 14 represent a graphical model of one embodiment of the process for haploid cells.

This approach is beneficial in the modelling of a complex stochastic system because it allows the “experts” to concentrate on the structure of the problem before having to deal with the assessment of quantitative issues. It is also appealing in that the model can be easily modified to incorporate other contributing factors to the process such as contamination. We provide a generalised model, but recognise that this can be continuously improved by modifying the nodes—for example, PCR efficiency is itself a variable that decreases with molecular weight of the target sequence, and this relationship can be also be easily modelled. π_{PCReff} is also affected by degradation where the high molecular weight material has preferentially degraded—but we envisage that the continued development of multiplexed real time quantification assays where PCR fragments of different sizes can be analysed will give a better indication of the degradation characteristics of the sample. Pre-casework assessment strategies informed by real time PCR quantitative assays such as the Applied Biosystems Quantifiler™ kit, combined with expert systems will remove much of the guess-work currently associated with DNA processing.

### Applications and Uses of the Invention

Once established the simulation can be used for a wide variety of purposes and to deliver a wide variety of benefits. Some examples are now provided.

### Use of the Simulation to Generate Random Profiles

Taking information from allele frequency databases, it is possible to use the simulation to generate random DNA profiles. These can be done on a very large scale as the time consuming and expensive physical analysis is not required.

By varying the parameters, for instance, that describe quantity and PCR efficiency, it is possible to simulate entire SGM plus profiles comprising 11 loci. At low quantities of DNA, stochastic effects result in partial DNA profiles. Consequently, each time a different PCR is carried out, each will give a different result. Either drop-out occurs, or samples are very unbalanced within and between loci. Some researchers have attempted to improve systems by using alternative amplification methods. In particular, there is much interest in Whole Genome Amplification. However, we have been able to quickly demonstrated through the use of the simulation that the reasons for imbalance are predominantly stochastic; and not related to biochemistry. Provided that nₜ>T a theoretical basis to improve profile morphology by applying a novel enzymatic biochemistry does not seem to exist simply because the allelic imbalance is predominantly a function of the number of molecules present at the start (n₀).

### Use of the Simulation to Run Mock Analyses

In the light of the issues mentioned in the previous section and given the generally applicable issues when there is limited DNA available to process, the invention offers further benefits.

Using the simulation it is possible simulate the starting point and DNA consideration process for mock samples so as to produce entirely simulated DNA profiles. By doing this before any analysis of the actual sample is performed useful information and warnings on issues effecting the process can be obtained. This assists in the decision making process for the analysis of the actual sample, in terms of the decisions on π_{aliquot} and the number of cycles (t) required to ensure nₜ>T, for instance.

### Use of the Simulation to Evaluate Issues

In a variation on the warnings prior to analysing a sample mentioned above, it is possible to quantify the impact of one or more issues on the sample and hence potentially direct a particular approach to its full analysis. In the context of degradation, for instance, it is possible to simulate the impact of degradation and potentially direct that the sample should be analysed using LCN, Low Copy Number analysis procedures where the degradation impact is particularly great and other approaches might not be successful as a result. In this respect the type of information outlined above in FIG. 18a, 18b, I& and 18d assists, as does the approaches to modelling then discussed. The discussion in that section of this document also makes it clear that the degradation impact should be considered in the context of the fragment size in question, rather than a different sized fragment which could potentially be far more protected against degradation.

### Use of the Simulation to Evaluate Different Processes

New methods of quantification that employ real time PCR analysis are much more accurate than those previously utilised, hence this also greatly assists the pre-assessment process and does make the DBA consideration process more powerful, especially when estimating N, n and π_{PCReff} parameters. In addition, methods that specifically amplify a portion of the Y chromosome are important to give an indication of the quantity and quality of the male DNA. Combining the Applied Biosystems Quantifiler™ and Y-Quantifiler™ tests therefore provides an opportunity to separately assess the male/female mixture components before the main test is actually carried out. Again all of these can be simulated using different simulations provided according to the invention. The simulations can consider the usefulness of those approaches to particular samples.

Furthermore, the ability to generate random profiles easily, with a full range of variability in the form and processing, allows the general usefulness of these processes to be considered and/or allows the format of those processes to be optimised in response to testing using the simulations. Development of these approaches are important because one of the biggest interpretational challenges is with mixtures (which are commonly encountered in forensics) and these approaches offer potential in that respect.

Previous development of such systems was dependent upon a direct assessment of the output data and could only be made after the cost had been incurred and time spent on real samples. In this invention the problem has been approached in a completely different way. Rather than analyse the output data from the electropherogram, a simulation is produced that includes input parameters n, N and π_{PCReff}. To this a Monte-Carlo simulation, for instance, is applied in order to determine, in a probabilistic way, a range of results. This is a much more powerful approach than those previously described, simply because the output parameters that generate the distributions of Hb, nₜ, p(D) and p(S) are crucially dependent upon the input parameters π_{extraction}, π_{PCReff}, n, N and t.

### Use of the Simulation to Improve Expert Systems

Following on from the issue in the passage above, it is not only parts of the DNA consideration process or new such processes which can be improved using the present invention. The approach can be used to feed enhanced information to and hence improve existing expert systems which currently use these generalised parameters in their software.

For example, to characterise mixtures, an algorithm called PENDULUM, Bill, M, P. Gill, et al. (2004). “PENDULUM-A guideline based approach to the interpretation of STR mixtures.” Forens. Sci. Int. in press. is used, based upon residual least squares theory. In PENDULUM Hb is generalised at 0.5 and a series of heuristics are used to interpret low level DNA profiles. Through the approach of the present invention it is possible to modify the parameters on a case-by-case basis and then import them into the final interpretation package, PENDULUM. Such information is provided in FIGS. 15, 16 and 17.

### Use of the Simulation to Consider Mixtures

The approach of the invention can equally well be used to generate random mixtures for any number of individuals. For example, to generate simple low copy number two person SGMplus male/female mixtures. The mixture proportion (Mx) of a male/female mixture, where there are nₘₐₗₑ and n_{female} input DNA molecules is defined as:

$$Mx = \frac{{n}_{male}}{{n}_{male} + {n}_{female}}$$

By repeatedly simulated pairs of SGM plus profiles, using defined n parameters to simulate a defined Mxᵢₙₚᵤₜ (which is the true mixture proportion) and then analysed the generated profiles with PENDULUM or other expert system enhanced information and results can be achieved. PENDULUM can be used to deconvolve the mixture back into the constituent contributors, ranking the first 500 results along with a density estimate of Mxₒᵤₜₚᵤₜ.

### Use of the Simulation to Consider Outlying Results

In existing processes, the majority of data may gave results that are easily interpreted. This is usually enough. However, the approach of the present invention renders it sufficiently easy to examine the behaviour of outliers that work on them is made easier. Indeed the simulation can even be set up so as to specifically generate profiles or information of such a nature. As a result it is possible to assess what may be reasonably expected during the course of casework; for example, how much can a PENDULUM estimate of Mx be affected by stochastic variation?

To demonstrate such an approach, the invention was used to simulate 1000 male/female LCN mixtures where Mxᵢₙₚᵤₜ=0.28 male. The most extreme example obtained, FIG. 15, resulted in highly unbalanced loci e.g. HUMVWA and HUMFTBRA/FGA, FIGS. 16 and 17, and yet PENDULUM was still able to deconvolve the mixture into its constituent genotypes.

This simple example illustrates that datasets produced by the invention are very powerful due to their being an unlimited amount of artificial, yet realistic, test-data. By providing case-specific input and output parameters to create probability distributions, this can subsequently be used to test robustness and to improve the functionality of external expert systems such as PENDULUM. To attempt to generate such data by conventional experimental means, by simultaneously varying all of the input parameters would not be feasible, or would be very time consuming, since literally thousands of physical experiments would be required to cover all possible combinations of parameters. We propose therefore that computer simulation is a useful tool to speed some of the more onerous tasks associated with validation of a new method.

### Use of the Simulation in New Approaches

Give the success of the invention in the above areas, work is now under way to demonstrate the approach in the context of Markov Chain Monte Carlo Methods to interpret mixtures. This is proposed on the basis of taking a casework result (by definition comprising an unknown number of contributors) and modelling results in the simulation by simultaneously and randomly varying all of the input parameters in order to arrive at a probabilistic evaluation of the evidence.

### Other Uses and Benefits of the Simulation

Generally speaking the approach of the invention is applicable to all DNA process considerations using STRs or SNPs or other methods. It is particularly beneficial where stochastic effects need to be measured. This includes medical and forensic applications.

Furthermore, the method has a universality such that it can be used to improve all aspects of the DNA processing laboratory. It can interact with any other expert system to accept input or output parameters and to provide test data. These benefits are due to the invention's ability to consider both inputs and outputs and there interrelationship as discrete parts. As a consequence, modifications, enhancements and simplifications can be made quickly and effectively without the need to change the system wholesale.

Developments Arising from the Use of the Simulation Approach

As well as the benefits of the simulation approach itself, the information it provides also enables-refinements and developments of existing techniques and concepts.

Two such developments stem from the investigation of degradation described above.

Firstly, the results detailed in FIGS. 19, 20 and 21 reveal significant information relevant to the selection of identity indicators to be investigated and to the adjacent fragments which are involved in their consideration. As discussed above, degradation occurs preferentially with respect to larger fragments compared with smaller fragments. The crucial inflexion or turning point is around 125 bases. Thus fragments of this size and less stand a greater chance of surviving degradation processes intact and hence amplifying and contributing to the revelation of their related identifier in any analysis approach. There is thus a clear pointer to the selection of fragments of 125 bases or less to be used and potentially even a pointer to the use of identifiers which can be investigated successfully using such fragment lengths. This is relevant to future analysis technique design. This position applies irrespective of the type of analysis approach used, but is particularly relevant to STR and mini-STR based approaches.

Secondly, the approach allows the improvement of existing technologies such as DNA sample quantification techniques.

The Quantifiler Human DNA quantification kit and/or Quantifiler Y Human Male DNA quantification kit (both available from Applied Biosystems, Foster City, Calif.) are intended to quantify the total amount of amplifiable DNA in a sample. Such an investigation allows a determination as to whether there is enough DNA to analyse and/or details of the analysis protocol to use. In the Quantifiler Human DNA quantification kit the target is the Human telomerase reverse transcriptase gene (hTERT) which is located at 5p15.33 and has an amplicon or fragment length of 62 bases. In the case of the Quantifiler Y Human Male DNA quantification kit the target is the Sex-determining region Y gene (SRY) which is located at Yp11.3 and has an amplicon or fragment length of 64 bases.

In both cases, a small aliquot of the sample to be quantified is taken and contact with a forward primer, reverse primer and probe. The probe has a fluorescent unit at the 5′ end and quencher unit at the 3′ end, which quenches the fluorescence of the fluorescent unit when that probe is intact. As the amplification progresses the extension of the forward primer cleaves the fluorescent unit from the probe and then displaces the quencher. The break up of the probe causes the florescent unit to fluoresce and this can be detected cycle by cycle as the amount of broken probes increases. Instruments, for instance provided with ABI Prism 7000 and 7900HT Sequence Detection System Software use the number of cycles required for the fluorescence level to cross a threshold to indicate the amount of amplifiable DNA present.

In both these specific cases the fragment used for the quantification process has a size of 62 or 64 bases. However, the present invention has revealed that such size fragments may be preferentially shielded from the effects of degradation. As a result, the amount of a fragment of size 62 bases in a sample may well not reflect the amount of a fragment of a larger size, say 150 bases. As a result the amount of quantifiable DNA may be an over estimate, particularly as 62 or 64 bases is well below the size at which protection against degradation occurs and/or when the different fragments being considered in the analysis are of predominantly of sizes larger than 125 bases.

The quantification techniques can be modified in a number of ways to address this issue.

Firstly, it would be possible to replace the small fragment being considered in such techniques with a fragment size which is more representative of the fragments of interest in the later analysis process and/or which is more exposed to degradation and hence would give a pessimistic answer to the amount of DNA rather than an optimistic one (a pessimistic answer may lead to an unnecessarily expensive or time consuming protocol being used to reach a proper result, but an optimistic answer may lead to the only sample being wasted on a protocol which does not provide a result).

Secondly, it would be possible to extend the quantification technique to base its quantification measurement on more than one fragment size. By providing the different probes for the different fragments with different fluorescent units (or other distinguishing units) it would be possible to simultaneously measure the amount of two or more different fragments. One of these could be the established 62 base or 64 base fragment, with another target being used which uses a larger fragment, say 200 bases or so. The result would be a better measure of the amount of amplifiable DNA present. The approach could be extended further to say a lower size fragment, 62 bases, fragment near the crucial size, say 125 bases, and fragment appreciably above the crucial size, say 200 bases.

In a further extension of this approach, the differences between the amounts of DNA indicated as present by the two or more different fragments can be used to provide information on the extent of degradation and potentially even the age of the sample. Thus at a short time after degradation could have possibly started, an equivalent quantity of DNA should be indicated for each fragment size. Once degradation has progressed, however, the 62 base suggested amount will not decrease as rapidly as the 125 base suggested amount, which will not decrease as rapidly as the 200 base suggested amount. Simulation and/or experimentation can be used to investigate and define the relationship of these variations with time. Hence for an unknown extent of degradation sample the differences can be used to identify the degradation extent.

1. A method of optimizing one or more parameters in a process for considering a DNA containing sample, the method comprising providing a computer implemented method of modeling the process for considering a DNA containing sample, the process being modeled by a graphical model, the model providing the one or more optimized parameters.

2. A method according to claim 1 wherein the method is used to determine the number of cells required for the process and/or to determine the extraction efficiency and/or to determine the sub-sample volume relative to the sample volume and/or to determine the amplification efficiency and/or to determine the optimum number of amplification cycles and/or to determine the effect of degradation on the amount of amplifiable DNA in the sample.

3. A method according to claim 1 wherein the method of modeling is used to model one or more test scenarios, the consideration process being modified in one or more ways as a result of the modeling.

4. A method according to claim 1, wherein the method of modeling is used to model one or more different processes under development, the process being modified as a result of the modeling.

5. A method according to claim 1, wherein the process for considering the DNA comprises extraction from the sample to provide an extracted sample, selection of a sub-sample of the sample, amplification of a sub-sample by PCR to give an amplified product, electrophoresis of the amplified product or a part thereof of the sub-sample, analysis of the sub-sample the analysis including allocation of allele designations.

6. A method according to claim 1, wherein the graphical model is formed of one or more nodes and one or more directed edges which extend between nodes.

7. A method according to claim 1, wherein the graphical model represents one or more of the parts of the process by a node, a node representing a parameter, with links between nodes representing the dependencies between parts of the process.

8. A method according to claim 1, wherein the model takes into account one or more parameters selected from: the number of cells in the sample; the proportion of the sample extracted into an extracted sample by the process; the extraction efficiency; the volume of the sub-sample relative to the volume of the sample the sub-sample is taken from; the amplification efficiency; the fraction of the amplifiable molecules amplified in each cycle of PCR; the number of cycles of amplification

9. A method according to claim 1, wherein the model takes into account one or more parameters selected from: the probability of allele dropout; the number of molecules of one or more of the alleles of interest after amplification; the ratio of the number of molecules of one allele compared with another for a locus; the heterozygous balance.

10. A method according to claim 1, wherein the model is used to model one or more of: allele dropout; allele dropout due to the absence of one or more allele types from the sample and/or extracted sample and/or sub-sample; allele dropout due to one or more allele types being below the detectable level in the amplification product; allele dropout due to stochastic effects; allele dropout due to degradation of the sample

11. A method according to claim 1, wherein the model is used to model stutter and/or contamination.

12. A method according to claim 1, wherein the method of modeling uses binomial theory to model one or more parts of the process.

13. A method according to claim 12 wherein the binomial theory is of the form Bin (n, π), where n is the number of template molecules for the part of the process and π is an efficiency parameter between 0-1 for that part of the process.

14. A method of modeling a process for considering a DNA containing sample, the process being modeled by a graphical model.

15. A method according to claim 14 wherein the method of modeling is used to improve one or more aspects of a DNA processing laboratory.
